SEQUENCE LISTING (42 sequences)

SEQ ID NO: 1 - RNA, 22 nt, Artificial Sequence
  miR-323a-5p
aggugguccguggcgcguucgc

SEQ ID NO: 2 - RNA, 19 nt, Artificial Sequence
  siRNA KIF11
cuagauggcuuucucagua

SEQ ID NO: 3 - RNA, 19 nt, Artificial Sequence
  siRNA KIFF11_as
uacugagaaagccaucuag

SEQ ID NO: 4 - RNA, 21 nt, Artificial Sequence
  siRNA INCEP_as
aguccuuuauuaagcgcaauu

SEQ ID NO: 5 - RNA, 21 nt, Artificial Sequence
  siRNA INCEP_as
aauugcgcuuaauaaaggacu

SEQ ID NO: 6 - RNA, 19 nt, Artificial Sequence
  siRNA_ CHAF1A
cugucauguggguucugac

SEQ ID NO: 7 - RNA, 19 nt, Artificial Sequence
  siRNA CHAF1A_as
gucagaacccacaugacag

SEQ ID NO: 8 - RNA, 19 nt, Artificial Sequence
  siRNA CDC25A
ggaaaaugaagccuuugag

SEQ ID NO: 9 - RNA, 19 nt, Artificial Sequence
  siRNA CDC25A_as
cucaaaggcuucauuuucc

SEQ ID NO: 10 - RNA, 19 nt, Artificial Sequence
  siRNA FADD
ugcguucuccuucucugug

SEQ ID NO: 11 - RNA, 19 nt, Artificial Sequence
  siRNA FADD_as
cacagagaaggagaacgca

SEQ ID NO: 12 - RNA, 21 nt, Artificial Sequence
  siRNA CCND1
ccuacgauacgcuacuauauu

SEQ ID NO: 13 - RNA, 21 nt, Artificial Sequence
  siRNA CCND1_as
aauauaguagcguaucguagg

SEQ ID NO: 14 - RNA, 19 nt, Artificial Sequence
  siRNA BCLX
caggcgacgaguuugaacu

SEQ ID NO: 15 - RNA, 19 nt, Artificial Sequence
  siRNA BCLX_as
aguucaaacucgucgccug

SEQ ID NO: 16 - protein, 7 aa, Artificial Sequence
  peptide
WHWRLPS

SEQ ID NO: 17 - protein, 18 aa, Artificial Sequence
  peptide
NGRGGVRSSSRTPSDKYC

SEQ ID NO: 18 - protein, 18 aa, Artificial Sequence
  peptide
CNGRCGVRSSSRTPSDKY

SEQ ID NO: 19 - protein, 18 aa, Artificial Sequence
  peptide
GNGRGGVRSSSRTPSDKY

SEQ ID NO: 20 - protein, 7 aa, Artificial Sequence
  peptide
CPRECES

SEQ ID NO: 21 - protein, 19 aa, Artificial Sequence
  peptide
CPRECESARSSSRTPSDKY

SEQ ID NO: 22 - protein, 7 aa, Artificial Sequence
  peptide
HSYWLRS

SEQ ID NO: 23 - protein, 14 aa, Artificial Sequence
  peptide
YSHSHSYWLRSGGG

SEQ ID NO: 24 - protein, 18 aa, Artificial Sequence
  peptide
RALKYSHSHSYWLRSGGG

SEQ ID NO: 25 - RNA, 19 nt, Artificial Sequence
  siControl 1
guaagacacgacuuaucgc

SEQ ID NO: 26 - RNA, 19 nt, Artificial Sequence
  siControl 1_as
cauucugugcugaauagcg

SEQ ID NO: 27 - RNA, 24 nt, Artificial Sequence
  miRNA Control 1
ucacaaccuccuagaaagaguaga

SEQ ID NO: 28 - DNA, 21 bp, Artificial Sequence
  primer
tcacccaattcatgaagaagc

SEQ ID NO: 29 - DNA, 20 bp, Artificial Sequence
  primer
gatcatacagtcgccctcct

SEQ ID NO: 30 - DNA, 20 bp, Artificial Sequence
  primer
gctgcgaagtggaaaccatc

SEQ ID NO: 31 - DNA, 22 bp, Artificial Sequence
  primer
cctccttctgcacacatttgaa

SEQ ID NO: 32 - DNA, 18 bp, Artificial Sequence
  primer
agctgtcatcgtgaagaa

SEQ ID NO: 33 - DNA, 21 bp, Artificial Sequence
  primer
cttggcgatcttcttcttgcc

SEQ ID NO: 34 - DNA, 42 bp, Artificial Sequence
  RNU44
cctggatgatgatagcaaatgctgactgaacatgaaggtctt

SEQ ID NO: 35 - protein, 15 aa, Artificial Sequence
  peptide
YSHSHSYWLRSGGGC

SEQ ID NO: 36 - protein, 17 aa, Artificial Sequence
  peptide
RCNPNMEPPRCWAAEGD

SEQ ID NO: 37 - protein, 17 aa, Artificial Sequence
  peptide
VCNPLTGALLCSAAEGD

SEQ ID NO: 38 - protein, 97 aa, Artificial Sequence
  peptide
MLGNKRLGLSGLTLALSLLVCLGALAEAYPSKPDNPGEDAPAEDMARYYSALRHYINLITRQRYGKRSSPETLISDLLMRESTENVPRTRLEDPAMW

SEQ ID NO: 39 - protein, 18 aa, Artificial Sequence
  peptide
CENLYFQSGSMAHPYFAR

SEQ ID NO: 40 - protein, 29 aa, Artificial Sequence
  peptide
YTIWMPENPRPGTPCDIFTNSRGKRASNG

SEQ ID NO: 41 - protein, 30 aa, Artificial Sequence
  peptide
KSVRTWNEIIPSKGCLRVGGRCHPHVNGGG

SEQ ID NO: 42 - protein, 15 aa, Artificial Sequence
  peptide
CCYHWKHLHNTKTFL

(19) United States
(12) Patent Application Publication
(10) Pub. No.: US 2022/0249376 A1
(43) Pub. Date: Aug. 11, 2022

(54) NANOVESICLES AND ITS USE FOR NUCLEIC ACID DELIVERY

(71) Applicants: FUNDACIÓ HOSPITAL UNIVERSITARI VALL D'HEBRON - INSTITUT DE RECERCA, BARCELONA (ES); CONSEJO SUPERIOR DE INVESTIGACIONES CIENTÍFICAS (CSIC), MADRID (ES); CONSORCIO CENTRO DE INVESTIGACIÓN BIOMÉDICA EN RED, M.P., MADRID (ES)

(72) Inventors: Miguel Francisco SEGURA GINARD, BARCELONA (ES); Soledad GALLEGO MELCON, BARCELONA (ES); Josep SÁNCHEZ DE TOLEDO CODINA, BARCELONA (ES); Aroa SORIANO FERNÁNDEZ, BARCELONA (ES); Nora VENTOSA RULL, BELLATERRA (ES); Jaume VECIANA MIRÓ, BELLATERRA (ES); Ariadna BOLOIX AMENÓS, BARCELONA (ES); Nathaly Verónica SEGOVIA RAMOS, TERRASSA (ES)

(21) Appl. No.: 17/610,971

(22) PCT Filed: May 12, 2020
(86) PCT No.: PCT/EP2020/063195

§ 371 Date: Nov. 12, 2021

(30) Foreign Application Priority Data
May 13, 2019 (EP) 19382372.1

(51) Int. Cl. A61K 9/127; A61K 31/713; C12N 15/11; C12N 15/88

(52) U.S. Cl. CPC A61K 9/1272; A61K 31/713; C12N 2320/32; C12N 15/88; C12N 2310/141; C12N 15/111

(57) ABSTRACT

The present invention refers to a nanovesicle comprising a sterol and a non-lipid cationic surfactant, for example myristalkonium chloride, wherein the sterol comprises DC-cholesterol. It also refers to a pharmaceutical composition that comprises it and its uses as a delivery system and as a bioimaging and theranostic tool. Furthermore, it also refers to the nanovesicle or the pharmaceutical composition for use as a medicament, in particular for use in the treatment of cancer.

This application is a U.S. National Stage Application under 35 U.S.C. § 371 of International Patent Application No. PCT/EP2020/063195 filed May 12, 2020, which claims the benefit of European Patent Application EP19382372.1 filed on May 13, 2019. Both of which are incorporated by reference in their entirety.

### TECHNICAL FIELD

The present invention relates in general to the field of nanovesicles which are useful in the delivery of nucleic acids, in particular small RNA. The present invention provides, among others, the nanovesicles, as well as a process for the preparation of these nanovesicles, and uses thereof in the treatment of diseases such as cancer (e.g. neuroblastoma).

### BACKGROUND ART

RNA therapeutics is an emerging field with a promising number of targets around all the transcriptome, which includes small RNAs like small interfering RNA (siRNA), microRNA (miRNA), among others (Bumcrot D et al. Nat Chem Biol 2006, 2:711-719). Although, the RNA based-therapies may be an alternative to chemoresistant tumours, the in vivo administration is still a challenge in the field, due to the rapid clearance and degradation of small RNAs in the bloodstream.

Nanovesicles have been the subject of numerous studies due to their potential use for encapsulating nucleic acids and drugs and to their applications in clinics. Among nanovesicles, liposomes are the most studied ones but recently increasing interest has been put on non-liposomal lipid nanovesicles. Quatsomes are non-liposomal lipid nanovesicles which are stable unilamelar nanovesicles with homogenous morphologies and which comprise quaternary ammonium surfactants, such as cetrimonium bromide (CTAB), myristalkonium chloride (MKC) or cetylpyridinium chloride (CPC), and sterols, such as cholesterol or β-sitosterol, in defined molar proportions (Grimaldi N. et al. Chem Soc Rev 2016, 45:6520-6545). Compressed fluid-based technologies such as Depressurization of an Expanded Liquid Organic Solution-Suspension method (DELOS-SUSP) have been used to produce Quatsomes (Grimaldi N. et al. Chem Soc Rev 2016, 45:6520-6545; WO2006079889).

Despite the promising usefulness of nanovesicles in nucleic acid delivery, they present a problem in the intracellular release of their content as they can become trapped in endosomes thus leading the nanovesicles to the degradation in the lysosomes and preventing their cargo content to be released in the cell cytoplasm.

From what it is known in the field, there is still a need to find a specific nucleic acid delivery method for diseases that use nucleic acids as therapeutic agent, such as cancer diseases, that effectively releases useful nucleic acids in the cell cytoplasm through escape from the endosome.

### SUMMARY OF INVENTION

The inventors have developed a tool for nucleic acid delivery, allowing the transported nucleic acid to perform its activity in the cytosol. This tool is a Quatsome (QS), which comprises non-lipid cationic surfactants (for example MKC) and DC-cholesterol (in a particular case is 100% DC-Chol), for example in a molar ratio 1:1, (from now on “the nanovesicle of the invention” or “the QS of the invention”).

The QS of the present invention are small unilamellar vesicles of less than 100 nm, low polydispersity, spherical shape and high colloidal stability over time (see FIGS. 1-4). Moreover, they are pH sensitive which allows buffering effect (see FIG. 5).

It was surprisingly found that DC-Chol formed nanovesicles when combined with a non-lipid cationic surfactant in all the formulations tested, whereas cholesterol or other cholesterol derivatives did not form always nanovesicles (FIG. 6 shows that the Chol-VS and the non-lipid cationic surfactant (CTAB) form ribbons; and FIG. 6C shows that in water with 10% of etOH cholesterol and the non-lipid cationic surfactant MKC formed preferably nanostructures like ribbons).

The inventors have used the nanovesicle of the invention for the siRNA and miRNA delivery in neuroblastoma cells with surprising results both in said nucleic acid expression and in the expression of their targets.

The QS of the present invention in comparison with QS that comprise other sterols have high RNA complexation efficiency (see FIG. 8), and high cellular viability when they are complexed with miRNA (see FIG. 9). The QS of the invention were, surprisingly, the only ones that when carrying a miRNA, also allowing its expression (see FIG. 10), they could modulate the expression of the targets of said miRNA (miR-323a-5p) at their mRNA level (see FIG. 11) and at their protein level (see FIG. 12) in neuroblastoma cells. The quatsome of the invention with 100% DC-Chol as the sterol (named “QS₄” in the examples below), was the best for miRNA delivery (see FIGS. 11 and 12). However, the miRNA release from QS is also produced at slow pace with the quatsome of the invention with nearly 50% DC-Chol as the sterols (named “QS₃” in the examples below) (see FIG. 13). The complexes QS₄-sRNA with the best efficacy in neuroblastoma cells for said effect were the complexes QS₄-miR-323a-5p (V) and QS₄-miR-323a-5p (VI), with a miRNA-to-QS₄ mass ratio 13.5·10⁻² and 20.24·10⁻² respectively (see table 7 and FIGS. 15-16). The transfection of miR-323a-5p with the QS of the invention reduced cell proliferation in a neuroblastoma cell line, with similar effects compared to Lipofectamine2000® (see FIG. 17-18). Transfection with siRNA was also possible with the QS of the invention, using siCCND1 in neuroblastoma cells (see FIGS. 19-22).

From the data provided below, it is remarkable the fact that the QS of the present invention can be used as nucleic acid delivery system for diseases that can be treated with nucleic acids, such as cancer, and for a particular example, neuroblastoma.

Moreover, it is demonstrated that the nanovesicles of the present invention protect the nucleic acid cargo from RNAse A degradation (FIG. 25).

The QS of the invention can be efficiently functionalized, for example, with fluorescent molecules for the in vitro and in vivo tracking of these particles (see example 5 for functionalization with Dil, see FIG. 23), with targeting units, like peptides or antibodies, for promoting “selective” delivery of biomolecules at the specific target site; or with stealth polymers, like poly-(ethylene glycol) (PEG), for improving their blood-circulation time (Cabrera I, et al. 2013 Nano Letters, 2013, 13(8), 3766-3774). The functionalization of QS with Dil did not alter the conjugation of miRNA or delivery and reduced neuroblastoma proliferation to the same exptent than non-functionalized QS-miRNA complexes (see FIG. 24).

Biodistribution analyses of QS₄ conjugated with miR-Control or miR-323a-5p showed that miR-323a-5p expression was increased in liver, lungs, spleen, kidney and subcutaneous tumors (150-, 66000-, 15000-, 570- and 125-fold, respectively) 24 h after a single administration and compared to QS₄-miRNA control (see FIG. 26). No macroscopic signs of toxicity nor adverse side effects were observed.

Thus a first aspect of the invention refers to a nanovesicle comprising a sterol and a non-lipid cationic surfactant, wherein the sterol comprises DC-cholesterol (DC-Chol).

A second aspect of the invention refers to a pharmaceutical composition comprising a therapeutically effective amount of the nanovesicle of the first aspect of the invention and a pharmaceutically acceptable excipient or vehicle.

A third aspect of the invention refers to the nanovesicle of the first aspect of the invention or the pharmaceutical composition of the second aspect of the invention as a delivery system.

The nanovesicle of the first aspect of the invention or the pharmaceutical composition of the second aspect of the invention can be used for the treatment of diseases that use nucleic acids as therapeutic agent, for example, for the treatment of human diseases.

A fourth aspect of the invention refers to the nanovesicle of the first aspect of the invention or the pharmaceutical composition of the second aspect of the invention for use as a medicament.

A fifth aspect of the invention refers to the nanovesicle of the first aspect of the invention or the pharmaceutical composition of the second aspect of the invention for use in the treatment of a non-infectious disease, preferably for the treatment of cancer.

A sixth aspect of the invention refers to the use of the nanovesicle of the first aspect of the invention as a bioimaging tool.

The preparation of QS can be performed by the CO₂-based DELOS-SUSP methodology (WO2006079889), which ensures a robustness and the reproducible scale up of QS which allows the preparation of nanomedicines in sufficient quantities for both preclinical and clinical testing.

A seventh aspect of the invention refers to a process for the production of the nanovesicle of the first aspect of the invention using the DELOS-SUSP methodology.

An eighth aspect of the invention refers to a kit comprising the nanovesicle of the first aspect of the invention or the pharmaceutical composition of the second aspect of the invention.

A ninth aspect of the invention refers to the use of the nanovesicle of the first aspect of the invention as a theranostic tool.

A tenth aspect of the invention refers to the nanovesicle of the first aspect of the invention as a pH buffering agent.

### BRIEF DESCRIPTION OF DRAWINGS

FIG. 1: Physicochemical properties of the indicated Quatsomes (QS) systems formed by the self-assembly of the quaternary ammonium surfactant (MKC) with different sterols (Chems, Cholesterol or DC-Chol). Hydrodynamic diameter and surface charge density of the indicated QS systems measured by DLS technique after one week (A) or two months (B) of Delos-SUSP preparation. Graph represents the mean±SD from the three independent experiments.

FIG. 2: Physicochemical properties of the indicated Quatsomes (QS) systems formed by the self-assembly of the quaternary ammonium surfactant (MKC) with different sterols (Chems, Cholesterol or DC-Chol). Hydrodynamic diameter and surface charge density of the indicated QS systems measured by DLS technique after one week (A) or two months (B) of nanovesicles purified by diafiltration. Graph represents the mean±SD from the three independent experiments.

FIG. 3: Narrow particle size distribution of various QS systems measured by DLS after (A) DELOS-SUSP preparation or (B) after diafiltration. Graph represents the mean±SEM of three independent experiments.

FIG. 4: High resolution representative Cryo-TEM images of QS varying the sterol composition. QS₀ (A), QS₁ (B), QS₂ (C), QS₃(D), QS₄ (E), QS₅ (F), QS₆ (G) and QS₇ (H). Scale barr, 200 nm.

FIG. 5: Buffering capacity of QS with different % of the pH sensitive sterol DC-Chol. The graph shows pH variations with an acidic HCl concentration from 0.01 μM to 3 μM.

FIG. 6: High resolution representative Cryo-TEM images of Chol/Chol-VS/CTAB aqueous mixture at different compositions: CS-VS₁: (32% Chol-VS/68% Chol):CTAB; CS-VS₂: (49% Chol-VS/51% Chol):CTAB; CS-VS₃: (66% Chol-VS/34% Chol):CTAB; CS-V54: (74% Chol-VS/26% Chol):CTAB; CS-VS₅ (100% Chol-VS/0% Chol):CTAB). A) High resolution representative Cryo-TEM images of CS-VS1 system taken 14 and 40 days after preparation. B) High resolution representative Cryo-TEM images of CS-V52, CS-V53, CS-VS4 and CS-VS₅ systems analyzed 14 days after sample preparation. C) High resolution cryo-TEM images representative of CS-CH composed of (100% Chol/0% DC-Chol):MKC nanostructures formed in water with 10% of EtOH at molar ratio 1:1 between the sterol and the surfactant after 7 days of sample preparation by DELOS-SUSP.

FIG. 7: Morphology and lamellarity of QS-miRNA complexes. High resolution representative Cryo-TEM images of different formulations of QS-miRNA complexes at various miRNA-to-QS mass ratios (III (A,D,G, J and M}; V (B, E, H, K and N}; VI(C, D, I, L and O}}, with different QS systems, QS₀ (A)-C}}, QS₁(D}-F}}, QS₂ (G}-1}}, QS₃(J)-L)) and QS₄ (M}-0)). Scale bar, 200 nm.

FIG. 8. Complexation efficiency of QS₄ with miRNAs by electrostatic interaction. Gel electrophoresis of miR-323a complexes with QS₄, at various miRNA-to-QS mass ratios (lanes 2-9), described in table 7, and standard calibration of naked miRNA (lane 11-14).

FIG. 9 High cell viability of QS and QS-miRNA complexes in chemoresistant NB cell lines (SK-N-BE(2)). Proliferation studies measuring the IC50 at 24 h post-incubation of QS (A) of QS₁₋₄-miR-control complexes (B). Mean±SEM is plotted from the duplicate experiments done.

FIG. 10: miR-323a-5p expression levels in SK-N-BE(2) cells transfected with miRNA naked (50 nM), micelles of MKC-miR-323a-5p at miRNA-to-MKC mass ratio (I) and QS-miR-323a-5p complexes at the indicated miRNA-to-QS mass ratios ((III), (IV), (V) and (VD), see table 7). miRNA expression levels were measured by qPCR. Graph represents the mean±SEM of three independent experiments. P<0.05*, p<0.01**, p<0.001***.

FIG. 11: Modulation of miR-323a direct targets after QS-miRNA complexes transfection. miRNA-direct target expression after miR-323a-5p or miR-Control (50 nM) transfection with naked miRNA, MKC micelles and the indicated QS systems in SK-N-BE(2) cells at 48 h. Graph represents the mean±SEM of three independent experiments. P<0.05*, p<0.01**, p<0.001***. All QS-miRNA complexes are described in table 7.

FIG. 12: Modulation of miR-323a direct targets at protein level after QS-miRNA complexes transfection. Representative band intensity quantification of the indicated proteins in NB cells, at 72 h post-transfection with the indicated QS-miRNA complexes. Histograms represent the quantification of band intensity signal mean±SEM from three independent experiments. P<0.05*, p<0.01**, p<0.001***. All QS-miRNA complexes are described in table 7.

FIG. 13: MiRNA release from QS₃ or QS₄ surface after overnight incubation with NB cells. Graphs shows the FRET ratio of ^{Dil}IQS-miR-Control^{Cy5} complexes with the different QS formulations after overnight transfection in SK-N-BE(2) neuroblastoma cells. All QS-miRNA complexes composition is described in table 7.

FIG. 14: Increased miR-323a-5p expression levels after QS₄-miR-323a-5p complexes transfection. miR-323a-5p expression levels in SK-NBE(2) measured by qPCR at 48 h post-transfection with QS₄-miR-323a-5p and with QS₄-miR-control complexes, both at the indicated miRNA-to-QS mass ratios (V, VI and VIII). Graph represents the mean±SEM of three independent experiments. P<0.05*, p<0.01**, p<0.001***. All QS-miRNA complexes are described in table 7.

FIG. 15: Modification of miR-323a-5p direct targets at mRNA expression level after 48 h post-transfection with QS₄-miR-323a-5p complexes in NB cells at the indicated miRNA-to-QS mass ratios (V, VI and VIII). Graphs represent the quantification of the mean±SEM of three independent experiments. P<0.05*, p<0.01**, p<0.001***. All QS-miRNA complexes are described in table 7.

FIG. 16: Modification of miR-323a-5p direct and indirect target at protein level after transfection with QS₄-miR-323a-5p complexes at the indicated miRNA-to-QS mass ratios (V, VI and VIII) in NB cells. Histograms represent the quantification of the mean±SEM of three independent experiments. P<0.05*, p<0.01**, p<0.001***. All QS-miRNA complexes are described in table 7.

FIG. 17: Reduction of SK-N-BE(2) cell proliferation after transfection with QS4-miR-323a-5p complexes at the indicated miRNA-to-QS mass ratios (I, III nad IV). Proliferation experiments were performed comparing miR-323a-5p versus miRNA-control (50 nM) complexed with QS₄ in NB cells at 96 h post-transfection. Graph represents the mean±SEM of three independent experiments. P<0.05*, p<0.01**, p<0.001***. All QS-miRNA complexes are described in table 7.

FIG. 18: Cell proliferation analysis in SK-N-BE(2) cells after transfection with QS4-miR-323a-5p complexes compared to Lipofectamine2000®). Proliferation experiments were performed comparing miR-323a-5p versus miRNA-control (50 nM) complexed with QS4 or liposomes (i.e. Lipofectamine 2000) in NB cells at 96 h post-transfection. Graph represents the mean of three independent experiments±SEM. P<0.05*, p<0.01**, p<0.001***. All QS-miRNA complexes are described in table 7.

FIG. 19: Modification of CCND1 direct target at mRNA expression level after 48 h post-transfection with QS₄-siCCND1 complexes at the indicated siRNA-to-QS mass ratios (V, VI nad VIII) in NB cells. Graph represents the mean±SEM of three independent experiments. P<0.05*, p<0.01**, p<0.001***. All QS-miRNA complexes are described in table 7.

FIG. 20: Modification of CCND1 direct and indirect targets modification at protein level after transfection with QS₄-siCCND1 complexes (V, VI and VIII) in NB cells. Histograms represent the quantification of the mean±SEM of three independent experiments. P<0.05*, p<0.01**, p<0.001***. All QS-miRNA complexes are described in table 7.

FIG. 21: Reduction of SK-N-BE(2) cell proliferation after transfection of QS₄-siCCND1 complexes (V, VI and VIII). Proliferation experiments were performed comparing siCCND1 versus siRNA-control (50 nM) complexed with QS₄ in NB cells at 96 h post-transfection. Graph represents the mean±SEM of three independent experiments. P<0.05*, p<0.01**, p<0.001***. All QS-miRNA complexes are described in table 7.

FIG. 22: Cell proliferation analysis in SK-N-BE(2) cells after transfection with QS4-siCCND1 complexes compared with Lipofectamine2000®. Proliferation experiments were performed comparing siCCND1 versus siRNA-control (50 nM) complexed with QS4 in NB cells at 96 h post-transfection. Graph represents the mean of three independent experiments±SEM. P<0.05*, p<0.01**, p<0.001***. All QS-miRNA complexes are described in table 7.

FIG. 23: Physicochemical properties of the indicated Quatsomes (QS₄) systems formed by the self-assembly of the quaternary ammonium surfactant (MKC) with DC-Chol sterol functionalized with Dil fluorophore (Dil-QS₄) or with the PEG stealth polymers (PEG-QS₄), composed of 10% Chol-PEG/90% DC-Chol:MKC. Hydrodynamic diameter and surface charge density of the indicated QS systems measured by DLS technique after one week of purification. Graph represents the mean of three independent experiments±SEM.

FIG. 24: Cell proliferation analysis of SK-N-BE(2) cells transfected with ^{Dil}QS₄-miR-323a-5p complexes and plain QS₄-miR-323a-5p complexes. Proliferation experiments were performed comparing miR-323a-5p versus miRNA-control (50 nM) complexed with ^{Dil}QS₄ or plain QS₄ in NB cells at 96 h post-transfection. Graph represents the mean of three independent experiments±SEM. P<0.05*, p<0.01**, p<0.001***. All QS-miRNA complexes are described in table 7.

FIG. 25. QS₄ protects miR-323a-5p from RNAse A degradation. Gel electrophoresis of miRNA protection after QS₄ complexation in RNAse A presence. QS₄ were loaded in lane 2, the QS₄-miRNA complexes at loading (V) (lane 3-8) and miRNA naked, as a negative control (lane 9-14). RNAse A (25 μg/mL) treatment complexes was done for thirty minutes, one hour, two or four hours (lane 5-12). SDS (0.25%) decomplexation was performed after complexes formation (lane 4), RNAse A treatment (lane 5-12) and in miRNA naked (lane 14).

FIG. 26: Biodistribution analysis ^{Dil}QS₄-miR-323a-5p complexes (2 mg/kg of miRNA with 10 mg/kg of QS4) were injected intravenously in athymic nude mice. Twenty four hours later, miR-323a-5p expression was analysed by qPCR. MiR-323a-5p was accumulated in subcutaneous neuroblastoma tumors, lungs, spleen, kidneys and liver compared to ^{Dil}QS₄-miR-Control injected mice. P<0.05*, p<0.01**, p<0.001**. QS-miRNA complexes preparation protocol is described in table 11.

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

DC-cholesterol, CAS number 137056-72-5, is also known as DC-Chol, Cholesteryl N-(2-dimethylaminoethyl)carbamate, or 3β-{N-[2-(Dimethylamino)ethyl]carbamoyl}Cholesterol or 3-(N—(N′, N′dimethylaminoethane)carbamoyl)cholesterol) or (C32-H56-N2-O2) or (cholest-5-en-3-ol (3beta)-, (2-(dimethylamino)ethyl)carbamate) or (3beta-(N—(N′, N′dimethylaminoethane)carbamoyl)cholesterol).

Non-lipid cationic surfactants include, but are not limited to, non-lipid cationic quaternary ammonium surfactants. The cationic surfactants of the present invention are not lipids.

Non-lipid quaternary ammonium surfactants are quaternary ammonium salts in which one nitrogen substituent is a long chain alkyl group. The non-lipid quaternary ammonium surfactants are water-soluble and self-assemble to form micelles above a critical micelle concentration (cmc). Conversely, the lipid quaternary ammonium surfactants self-assemble to form other structures, such as vesicles, planar bilayers or reverse micelles. The quaternary ammonium surfactants of the present invention are not lipids.

In an embodiment of the first aspect of the invention, the non-lipid cationic quaternary ammonium surfactant is selected from the list consisting of: myristalkonium chloride (MKC), cetyl trimethylammonium bromide (CTAB), cetylpyridinium chloride (CPC), benzalkonium chloride (BAC), cetyl trimethylammonium chloride (CTAC), benzethonium chloride (BZT), stearalkonium chloride, cetrimide, benzyldimethyldodecylammonium chloride, and combinations thereof.

In an embodiment of the first aspect of the invention, the non-lipid cationic quaternary ammonium surfactant is myristalkonium chloride (MKC).

Myristalkonium chloride (MKC), CAS number 139-08-2, is also known as benzyldimethyltetradecylammonium chloride or myristyldimethylbenzylammonium chloride or N-benzyl-N-tetradecyldimethylammonium chloride or N, N-dimethyl-N-tetradecylbenzenemethanaminium chloride or tetradecylbenzyldimethylammonium chloride.

In another embodiment of the first aspect of the invention, the non-lipid cationic quaternary ammonium surfactant is cetyl trimethylammonium bromide (CTAB).

The first aspect of the invention refers to a nanovesicle comprising a sterol and a non-lipid cationic surfactant wherein the sterol comprises DC-cholesterol (DC-Chol).

In an embodiment of the first aspect of the invention the sterol comprises DC-Chol in at least 5%, for example: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 77, 78, 79, 80, 81, 82, 83, 84, 85, 86, 87, 88, 89, 90, 91, 92, 93, 94, 95, 96, 97, 98, 99 or 100%.

In an embodiment of the first aspect of the invention the percentage of DC-Chol in respect to the total sterol is at least 20%, or, alternatively, at least 47%, or, alternatively, at least 90%, or, alternatively, 100%.

In an embodiment of the first aspect of the invention the sterol is a mixture of DC-Chol and cholesterol, or, alternatively, DC-chol and cholesterol derivatives. For example, the cholesterol derivative comprises polyethylene glycol (PEG). For example, a cholesterol derivative is Chol-PEGn-X wherein “n” is the length of the PEG chain (for example, n=0, or at least 1); and wherein “X” is —SH, —OH, —CHO, —OCH₃, —NH₂, —NH, —CH₃, —N₃, —COOH, -Maleimide, a peptide, an antibody or a sugar. Wherein the peptides can be selected from the list consisting of: a HSYWLRS peptide (SEQ ID NO: 22) (for example, sequence: YSHSHSYWLRSGGGC (SEQ ID NO: 35)), GD2 mimic binding peptide (for example, sequence: RCNPNMEPPRCWAAEGD (SEQ ID NO: 36) or VCNPLTGALLCSAAEGD (SEQ ID NO: 37)), neuropeptide Y (for example, sequence: MLGNKRLGLSGLTLALSLLVCLGALAEAYPSKPDNPGEDAPAEDMARYYSALRHYINLITRQRYGKRSSPETLI SDLLMRESTENVPRTRLEDPAMW (SEQ ID NO: 38)); a P75 neurotrophin receptor (for example, sequence: CENLYFQSGSMAHPYFAR) (SEQ ID NO: 39), a Rabies virus glycoprotein (RVG) peptide (for example, sequence: YTIWMPENPRPGTPCDIFTNSRGKRASNG (SEQ ID NO: 40) or KSVRTWNEIIPSKGCLRVGGRCHPHVNGGG) (SEQ ID NO: 41), a dopaminergic peptide (for example, sequence: CCYHWKHLHNTKTFL) (SEQ ID NO: 42), a RGD-peptide, and a GD2 antibody. Examples of sugars can be: D-glucose or glucosamine derivatives.

In an embodiment of the first aspect of the invention the nanovesicle is a non-liposomal lipid nanovesicle. In another embodiment of the first aspect of the invention the nanovesicle is a quatsome comprising 100% DC-Chol as the sterol and MKC at a ratio molar in the range of 10:1 to 1:5.

In an embodiment of the first aspect of the invention the nanovesicle is a quatsome comprising 100% DC-Chol as the sterol and MKC at a ratio 1:1. In another embodiment of the first aspect of the invention the nanovesicle is a quatsome comprising 100% DC-Chol as the sterol and MKC at a ratio 1:2 and 2:1.

In an embodiment of the first aspect of the invention the nanovesicle is spherical, unilamellar, homogeneous in size and stable.

There are well-known methods in the state of the art to characterize the nanovesicles of the invention, for example by means of their potential Z. The size of the nanovehicle can be measured by any method known to the expert, for example by dynamic light scattering (DLS), mass spectrometry, Small-angle X-ray scattering (SAXS), transmission electron microscopy (TEM) or high resolution transmission electron microscopy (HR-TEM).

For example, to characterize the nanovesicles of the invention the protocol disclosed in Danaei, M.; et al. “Impact of Particle Size and Polydispersity Index on the Clinical Applications of Lipidic Nanocarrier Systems” Pharmaceutics 2018, 10, 57, is followed.

In the present invention the term “spherical” refers to a diameter of 20-500 nm, for example between 50-300 nm.

The term “homogeneous size” refers to a nanovesicle with a polydispersity index (PDI) of 0.1-0.5, for example between 0.1-0.3.

The stablility of the nanovesicles of the present invention can be measured by dynamic light scattering (DLS).

For example, the stablility over time of the nanovesicles of the present invention can be measured by DLS and refers to a hydrodinamic diameter that upon time remains smaller than 300 nm and to a PDI in the range of 0.1-0.3.

In an embodiment of the first aspect of the invention the nanovesicle has a mean diameter smaller than 300 nm, a PDI of 0.1-0.3, and is stable at least up to 2 months.

In an embodiment of the first aspect of the invention the nanovesicle comprises a nucleic acid, i.e. a small RNA such as a miRNA, a siRNA or shRNA.

In an embodiment of the first aspect of the invention the nucleic acid is inside the nanovesicle. In another embodiment of the first aspect of the invention the nucleic acid is outside the nanovesicle.

The term “small RNA” refers to RNAs of less than 200 nucleotides in length. They are usually non-coding RNA molecules which are modulators of gene expression, for example microRNA (miRNA) or small interfering RNA (siRNA).

In an embodiment of the first aspect of the invention the nanovesicle comprises a nucleic acid which has a tumour suppressor function.

In an embodiment of the first aspect of the invention the miRNA is selected from the list consisting of: hsa-miR-323a-5p, hsa-miR-497, has-miR-380-5p, hsa-miR-892b, hsa-miR-654-5p, hsa-miR-885-3p, hsa-miR-193a-3p, hsa-miR-661, hsa-miR-491-3p, hsa-miR-193b-5p, hsa-miR-3150a-3p, hsa-miR-744-5p, hsa-miR-326, hsa-miR-665, hsa-miR-185-3p, hsa-miR-34b-5p, hsa-miR-138-2-3p, hsa-miR-4440, hsa-miR-450b-3p, hsa-miR-1180, hsa-miR-3140-3p, hsa-miR-4291, hsa-miR-30b-3p, hsa-miR-541-3p, hsa-miR-483-5p, hsa-miR-4292, hsa-miR-124-3p, hsa-miR-1207-5p, hsa-miR-193b-3p, hsa-miR-221-5p, hsa-miR-3913-3p, hsa-miR-5095, hsa-miR-891b, hsa-miR-1275, hsa-miR-299-3p, hsa-miR-149-3p, hsa-miR-132-5p, hsa-miR-509-3-5p, hsa-miR-3677-3p, hsa-miR-876-3p, hsa-miR-940, hsa-miR-4655-5p, hsa-miR-555, hsa-miR-342-5p, hsa-miR-3181, hsa-miR-3154, hsa-miR-5585-3p, hsa-miR-708-5p, hsa-miR-3135a, hsa-miR-4664-3p, hsa-miR-4289, hsa-miR-135a-3p, hsa-miR-522-5p, and any combinations thereof.

In an embodiment the miRNAs indicated above are the following according to the identification number of the public data base miRBase (at the date of 30 Apr. 2019): MIMAT0004696, MIMAT0002820, MIMAT0000734, MIMAT0004918, MIMAT0003330, MIMAT0004948, MIMAT0000459, MIMAT0003324, MIMAT0004765, MIMAT0004767, MIMAT0000734, MIMAT0015023, MIMAT0004945, MIMAT0000756, MIMAT0004952, MIMAT0004611, MIMAT0000685, MIMAT0004596, MIMAT0018958, MIMAT0004910, MIMAT0026735, MIMAT0015008, MIMAT0016922, MIMAT0004589, MIMAT0004920, MIMAT0004761, MIMAT0016919, MIMAT0000422, MIMAT0005871, MIMAT0002819, MIMAT0004568, MIMAT0019225, MIMAT0020600, MIMAT0004913, MIMAT0005929, MIMAT0000687, MIMAT0004609, MIMAT0004594, MIMAT0004975, MIMAT0018101, MIMAT0004925, MIMAT0004983, MIMAT0019721, MIMAT0003219, MIMAT0004694, MIMAT0015061, MIMAT0015028, MIMAT0022286, MIMAT0004926, MIMAT0015001, MIMAT0019738, MIMAT0016920, MIMAT0004595, MIMAT0005451, and any combinations thereof.

In an embodiment of the first aspect of the invention the miRNA is hsa-miR-323a-5p. In another embodiment of the first aspect of the invention the miRNA is SEQ ID NO: 1.

In an embodiment of the first aspect of the invention the siRNA is selected from the list consisting of: siCCND1, siCHAF1A, siINCENP, siKIF11, siCDCl25A, siFADD and siBCL-XL.

In an embodiment of the first aspect of the invention the siRNAs indicated above are siRNA that silence the expression of the following genes according to the identification number of the public data base Genbank (at the date of 30 Apr. 2019): the Gene ID 3832 (KIF11, kinesin family member 11), Gene ID 3619 (INCENP, inner centromere protein), Gene ID 10036 (CHAF1A, chromatin assembly factor 1 subunit A), Gene ID 993 (CDC25A, cell division cycle 25A), Gene ID 8772 (FADD, Fas associated via death domain), Gene ID 595 (CCND1, cyclin D1), and Gene ID 598 (BCL-XL, BCL2 like 1 isoform).

In an embodiment of the first aspect of the invention the siRNAs indicated above are selected from the list consisting of: SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, and any combinations thereof.

In an embodiment of the first aspect of the invention the siRNAs are SEQ ID NO: 2 and/or SEQ ID NO: 3; or, alternatively, SEQ ID NO: 4 and/or SEQ ID NO: 5; or, alternatively, SEQ ID NO: 6 and/or SEQ ID NO: 7; or, alternatively, SEQ ID NO: 8 and/or SEQ ID NO: 9; or, alternatively, SEQ ID NO: 10 and/or SEQ ID NO: 11; or, alternatively, SEQ ID NO: 12 and/or SEQ ID NO: 13; or, alternatively, SEQ ID NO: 14 and/or SEQ ID NO: 15.

In an embodiment of the first aspect of the invention the siRNA is siCCND1. In another embodiment of the first aspect of the invention the siRNA is the siCCND1 of sequence SEQ ID NO: 12.

In an embodiment of the first aspect of the invention the miRNA-to-QS mass ratio is comprised between and including 1×10⁻² to 300×10⁻², in another embodiment it is between and including 1×10⁻² to 100×10⁻², in another embodiment is between and including 1×10⁻² to 90×10⁻²; in another embodiment is 2×10⁻², 3×10⁻², 4×10⁻², 5×10⁻², 6×10⁻², 7×10⁻², 8×10⁻², 9×10⁻², 10×10⁻², 20×10⁻², 30×10⁻², 40×10⁻², 50×10⁻², 60×10⁻², 70×10⁻², 80×10⁻², 81×10⁻², 82×10⁻², 83×10⁻², 84×10⁻², 85×10⁻², 86×10⁻² or 87×10⁻².

In an embodiment of the first aspect of the invention the nanovesicle is further bound to an element selected from the group consisting of: a fluorophore, a radiopharmaceutical, a peptide, a polymer, an inorganic molecule, a lipid, a monosaccharide, an oligossacharide, an enzyme, an antibody or fragment of an antibody, an antigen, and any combination thereof.

In an embodiment of the first aspect of the invention the fluorophore is a carbocyanine fluorophore.

In another embodiment of the first aspect of the invention the carbocyanine fluorophore is 1,1′-dioctadecyl-3,3,3′,3′-tetramethylindocarbocyanine perchlorate.

In another embodiment of the first aspect of the invention the radiopharmaceutical is metaiodobenzylguanidine (MIBG) labelled with ¹³¹I.

In another embodiment of the first aspect of the invention the nanovesicle is bound to a hydrophilic polymer that prevents the opsonisation process (“stealth” polymer). In another embodiment of the first aspect of the invention the polymer is polyethilenglycol (PEGn).

In an embodiment of the first aspect of the invention the nanovesicle is bound to a tumor targeting peptide. In an embodiment of the first aspect of the invention the peptide is capable of recognizing cancer cells, such as neuroblastoma cells, and/or tumor-associated endothelial cells, for example WHWRLPS (SEQ ID NO: 16) peptides, NGR-containing peptides and RGD peptides, aminopeptidase A (glutamyl-aminopeptidase, APA) binding peptides or

In an embodiment of the first aspect of the invention, the NGR-containing peptides are the peptides SEQ ID NO: 17 (NGRGGVRSSSRTPSDKYC), SEQ ID NO: 18 (CNGRCGVRSSSRTPSDKY) or SEQ ID NO: 19 (GNGRGGVRSSSRTPSDKY).

The RGD peptides (comprising the Arg-Gly-Asp motif) are peptides commonly described in the art as peptides that are able to interact with integrins present in the membrane of cells, and of particular interest for the study of cell adhesion, both between cells and between cells and different tissues or the basement membrane.

Aminopeptidase A (glutamyl-aminopeptidase, APA) is a membrane-spanning cell surface protein overexpressed in angiogenic blood vessels and in perivascular cells of human tumors. In an embodiment of the first aspect of the invention, the APA-binding peptide is a peptide comprising the sequence CPRECES (SEQ ID NO: 20). In another embodiment of the first aspect of the invention, the APA-binding peptide is the peptide CPRECESARSSSRTPSDKY (SEQ ID NO: 21).

In an embodiment of the first aspect of the invention, the tumor targeting peptides are HSYWLRS-containing peptides (SEQ ID NO: 22), for example YSHSHSYWLRSGGG (SEQ ID NO: 23), RALKYSHSHSYWLRSGGG (SEQ ID NO: 24) or YSHSHSYWLRSGGGC (SEQ ID NO: 35).

In an embodiment of the first aspect of the invention the tumor targeting peptide is bound to PEG.

A second aspect of the invention refers to a pharmaceutical composition comprising a therapeutically effective amount of the nanovesicle of the first aspect of the invention and a pharmaceutically acceptable excipient or vehicle.

The expression “therapeutically effective amount” as used herein, refers to the amount of a compound (i.e. nanovesicle of the invention) that, when administered, is sufficient to prevent development of, or alleviate to some extent, one or more of the symptoms of the disease which is addressed. The particular dose of compound administered according to this invention will of course be determined by the particular circumstances surrounding the case, including the compound administered, the route of administration, the particular condition being treated, and the similar considerations.

The expression “pharmaceutically acceptable excipients or carriers, or vehicles” refers to pharmaceutically acceptable materials, compositions or vehicles. Each component must be pharmaceutically acceptable in the sense of being compatible with the other ingredients of the pharmaceutical composition. It must also be suitable for use in contact with the tissue or organ of humans and animals without excessive toxicity, irritation, allergic response, immunogenicity or other problems or complications commensurate with a reasonable benefit/risk ratio.

A third aspect of the invention refers to the nanovesicle of the first aspect of the invention or the pharmaceutical composition of the second aspect of the invention as a delivery system. This aspect can be reformulated as the use of the nanovesicle of the first aspect of the invention or the pharmaceutical composition of the second aspect of the invention as a delivery system.

In an embodiment of the third aspect of the invention the delivery system is a drug delivery system.

In an embodiment of the third aspect of the invention the delivery system is a drug delivery system for gene and/or epigenetic therapy, or for miRNA or siRNA transfection.

In an embodiment of the third aspect of the invention the delivery system is a nucleic acid transfect agent.

A fourth aspect of the invention refers to the nanovesicle of the first aspect of the invention or the pharmaceutical composition of the second aspect of the invention for use as a medicament.

The fourth aspect of the invention can be reformulated as the use of the nanovesicle of the first aspect of the invention or the pharmaceutical composition of the second aspect of the invention for the manufacture of a medicament.

A fifth aspect of the invention refers to the nanovesicle of the first aspect of the invention or the pharmaceutical composition of the second aspect of the invention for use in the treatment of cancer.

In an embodiment of the fifth aspect of the invention the cancer is neuroblastoma.

The fifth aspect of the invention can be reformulated as the use of the nanovesicle of the first aspect of the invention or the pharmaceutical composition of the second aspect of the invention for the manufacture of a drug for the treatment of a cancer disease, for example neuroblastoma. It can also be reformulated as a method for the treatment or prevention of a cancer disease, for example neuroblastoma, that involves administering a therapeutically effective amount of the first aspect of the invention's nanovesicle, together with pharmaceutically acceptable carriers or excipients, to a subject in need of it, including a human.

The medicament can be presented in a form adapted for parenteral, cutaneous, oral, epidural, sublingual, nasal, intrathecal, bronchial, lymphatic, rectal, transdermal or inhaled administration. The form adapted to parenteral administration refers to a physical state that can allow its injectable administration, that is, preferably in a liquid state. Parenteral administration can be carried out by intramuscular, intraarterial, intravenous, intradermal, subcutaneous or intraosseous administration, but not limited to these types of parenteral routes of administration. The form adapted to oral administration is selected from the list comprising, but not limited to, drops, syrup, tisane, elixir, suspension, extemporaneous suspension, drinkable vial, tablet, capsule, granulate, stamp, pill, tablet, lozenge, troche or lyophilized. The form adapted to rectal administration is selected from the list comprising, but not limited to, suppository, rectal capsule, rectal dispersion or rectal ointment. The form adapted to the transdermal administration is selected from the list comprising, but not limited to, transdermal patch or iontophoresis.

In an embodiment of the fourth and fifth aspects of the invention the medicament is presented in a form adapted for intravenous administration. In another embodiment of the fourth and fifth aspects of the invention the medicament is presented in a form adapted for oral administration.

In an embodiment of the fourth or fifth aspect of the invention the medicament is administered twice a week. In another embodiment of the fourth of fifth aspect of the invention the medicament is administered at least every 6, 8, 12, 24, 48 hours. In another embodiment of the fourth of fifth aspect of the invention the medicament is administered at least once a week or twice a week.

In an embodiment of the fourth and fifth aspects of the invention the medicament comprises a therapeutical amount of the nanoparticle of the first aspect of the invention, for example 10 to 30 μM miRNA, in another example is 15 to 20 μM miRNA, in yet another example is 17.7 μM miRNA for the administration in mice (which equals the 0.25 mg/mL and 2 mg/kg in mice). In another embodiment of the fourth and fifth aspects of the invention the medicament comprises a therapeutical amount of the nanoparticle of the first aspect of the invention of 0.2 to 3 mg/kg miRNA in humans, in another example is 0.3 to 2 mg/kg miRNA, in yet another example is 0.27 μM miRNA.

Advantageously, the nanovesicle of the first aspect of the invention can be easily functionalized, for example with fluorescent dyes to be observed by super-resolution microscopy (for example as described in Ardizzone et al, SMALL, 2018, 14). These fluorescent-nanovesicles when conjugated to fluorescent microRNA show fluorescence resonance energy transfer (FRET) signal, which can be used for tracking QS-miRNA cellular internalization and subcellular distribution. The nanovesicle of the first aspect of the invention can be used as a bioimaging tool to track nucleic acid internalization and delivery.

The nanovesicles of the present invention can be labelled, for example with a dye; and functionalized with targeting ligand for site-specific labelling; and finally deliver the therapeutic agent (for example, miRNA and/or siRNA).

Thus, a sixth aspect of the invention refers to the use of the nanovesicle of the first aspect of the invention as a bioimaging tool.

In an embodiment of the sixth aspect of the invention it is used as a bioimaging tool, to track nucleic acid (for example miRNA or siRNA) internalization and delivery.

As “bioimaging tool” is to be understood according to this description a reagent used in an imaging technique used in biology to trace some compartments of cells or particular tissues. Examples of bioimaging tools include chemiluminescent compounds, fluorescent and phosphorecent compounds, X-ray or alpha, beta, or gamma-ray emmiting compounds, etc.

The nanovesicle of the first aspect of the invention can, for example, be formed by self-assembly of the DC-Chol and the non-lipid cationic surfactant (i.e. MKC).

The nanovesicle of the first aspect of the invention can be formed by different techniques, such as ultrasonication (US), thin film hydration (THF) and a one-step scalable method using CO₂ expanded solvents called Depressurization of an Expanded Liquid Organic Solution-suspension (DELOS-susp) (WO2017147407; Cano-Sarabia M et al. Langmuir 2008, 24, 2433-2437; Elizondo E et al. Nanomed. 2012, 7, 1391-1408).

A seventh aspect of the invention refers to a process for the production of a nanovesicle of the first aspect of the invention using the DELOS-SUSP methodology.

In an embodiment of the seventh aspect of the invention, the DELOS-SUSP methodology comprises:
a) the preparation of an aqueous solution of the non-lipid cationic surfactant (i.e. MKC),
b) the dissolution of the DC-Chol in an organic solvent and then expanding the solution by using a compressed fluid (CF), and
c) the synthesis of the nanovesicles by despressurization of the resulting solution from step b) on the solution resulting from step a).

In another embodiment of the seventh aspect of the invention, wherein the DELOS-SUSP methodology comprises:
a) providing an aqueous solution,
b) the dissolution of the DC-Chol and the non-lipid cationic surfactant (i.e. MKC) in an organic solvent and then expanding the solution by using a compressed fluid (CF), and
c) the synthesis nanovesicles by despressurization of the resulting solution from step b) on the aqueous solution of step a).

In an embodiment of the seventh aspect of the invention, in order to prepare fluorescent nanovesicles comprising non-water soluble organic dyes, the method further comprises:

in step b) the dissolution of the DC-Chol and a non-water soluble organic dye in an organic solvent and then expanding the solution by using a compressed fluid (CF); and
in step c) fluorescent nanovesicles synthesis by despressurization of the resulting solution from step b).

Another aspect of the present invention is also the nanovesicle obtainable by method of the seventh aspect of the invention.

An eighth aspect of the present invention refers to a kit comprising the nanovesicle of the first aspect of the invention or the pharmaceutical composition of the second aspect of the invention. The kit can also comprise instructions for the delivery of the nucleic acid comprised in the nanovesicle of the first aspect of the invention. The kit may additionally comprise further means to visualize the nanovesicles.

Also part of the invention is the use of the kit of the eighth aspect of the invention for the uses described in the other aspects above or below of the present invention.

Also part of the invention is a kit comprising a device for release of the nanovesicle from the first aspect of the invention or the pharmaceutical composition from the second aspect of the invention and also comprising the nanovesicle from the first aspect of the invention or the pharmaceutical composition from the second aspect of the invention.

Also part of the invention is a device for the release of the nanovesicle from the first aspect of the invention or the pharmaceutical composition of the second aspect of the invention comprising them.

The nanovesicles of the first aspect of the invention is able to simultaneously diagnose, image, and treat targeted diseased sites, with a precise spatio-temporal control of the dosage while monitoring the treatment therapeutic efficiency. Therefore, a ninth aspect of the present invention refers to the use of the nanovesicle of the first aspect of the invention as a theranostic tool.

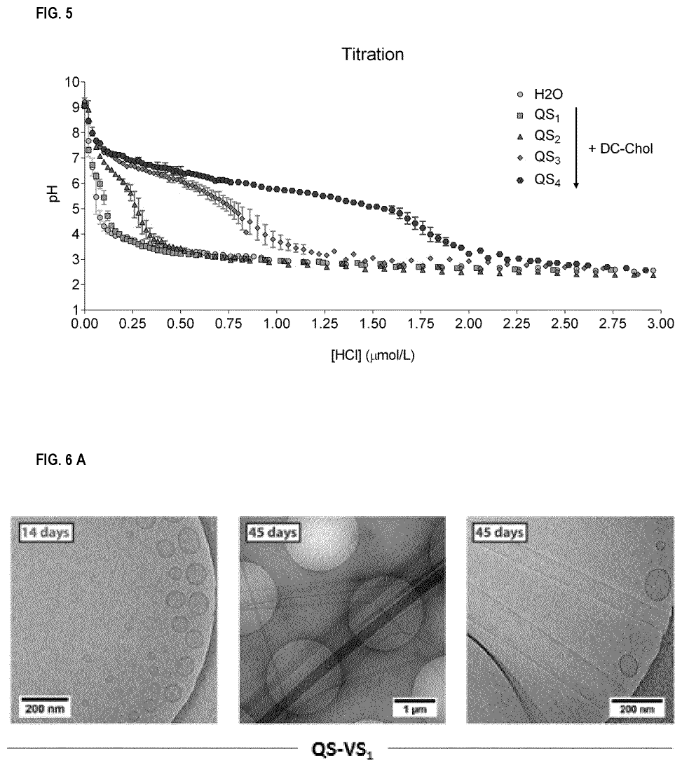

According to the present invention, the nanovesicles of the first aspect of the invention exhibit pH buffering capacity (see FIG. 5). Thus, a tenth aspect of the invention refers to the nanovesicle of the first aspect of the invention or the pharmaceutical composition of the second aspect of the invention as a pH buffering agent.

Another aspect of the present invention is the use of the nanovesicle of the first aspect of the invention or the pharmaceutical composition of the second aspect of the invention as an antibacterial agent or, alternatively, as an antifungal agent.

As antibacterial action of the nanovesicle of the first aspect of the invention or the pharmaceutical composition of the second aspect of the invention can be performed through the perturbation of the bacterial plasma membrane, for example, causing bacterial cell lysis. The antibacterial action can be measured by method known by the expert in the field, such as biofilm model, Alamar Blue assay measuring bacterial viability or with crystal violet stain; and can be proven in Gram positive or Gram negative bacteria; for example in known model pathogens such as Staphylococcus aureus, Bacillus subtillis or Escherichia coli.

The antibacterial action can be measured by method known by the expert in the field for example against Aspergillus niger or Candida albicans.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Reference signs related to drawings and placed in parentheses in a claim, are solely for attempting to increase the intelligibility of the claim, and shall not be construed as limiting the scope of the claim. Furthermore, the present invention covers all possible combinations of particular and preferred embodiments described herein.

### EXAMPLES

### 1. Quatsomes Synthesis

### 1.1 Quatsomes Synthesis and Physicochemical Characterisation

### Materials and Methods

Cholesten-3β-ol (Choi, purity 95%; #A0807; CAS n°: 57-88-5) and Sodium hydroxide (NaOH, purity 98.0%) were obtained from PanReac (Castellar del Valles, Spain). Cholesteryl N-(2-dimethylaminoethyl)carbamate (DC-Chol, purity 98%; #92243) and Cholesteryl hemisuccinate (Chems, purity 98%; #06512; CAS n°: 1510-21-0) were purchased from Sigma-Aldrich (Saint Louis, Mo., USA). Benzyldimethyltetradecylammonium Chloride (MKC; purity 99%; #262393) was supplied by AttendBio Research SL (Santa Coloma de Gramenet, Spain). Cetyltrimethylammonium bromide (CTAB, ultra for molecular biology) was purchased from Fluka-Aldrich. 1,1′-dioctadecyl-3,3,3′,3′-tetramethyl-indocarbocyanine perchlorate (Dil) was supplied by Thermofisher. Ethanol was purchased from Teknochroma (Sant Cugat del Vallès, Spain). The Polyethyleneglycol derivatives of cholesterol (mPEG-CLS; mPEG chain: 1000; #MF001095-1K) were purchased from BioChemPEG (Watertown, Mass. 02472, USA). Carbon dioxide (purity 99.9%) was acquired from Carburos Metálícos S. A. (Cornelia de Llobregat, Spain). All the chemicals were used without further purification and all solutions were prepared using pre-treated Milli-Q water (Millipore Ibérica, Madrid, Spain).

Lipofectamine 2000 (#11668019) were purchased from ThermoFisher Sientific (Waltham, Mass., USA).

Human synthetic miRNA mimics, Dy547 labelled-miR-Control-1 (#CP-004500-01; Table 1) and hsa-miR-323a-5p (#CP-301085-01; Table 1) were acquired from present Dharmacon Inc (Lafayette, Colo., USA). Control siRNA (5′ GUAAGACACGACUUAUCGC 3′) (SEQ ID NO: 25) and siCCND1 (5′ CCUACGAUACGCUACUAUAUU 3′) (SEQ ID NO: 12) were purchased from Sigma (Table 2).

TABLE 1
miR-Control and microRNA hsa-miR-323a-5p

| | miRNA | | |
|---|---|---|---|
| | Mimics | | |
| miRNA mimic | Catalog No | Sequence (Accession) | A/E maximum |
| miRIDIAN microRNA | CN-001000-01 | UCACAACCUCCUAGAAAGAGUAGA | — |
| Mimic Negative | | (MIMAT0000039) | |
| Transfection | | (SEQ ID NO: 27) | |
| Control 1 | | | |
| miRIDIAN microRNA | CP-004500-01 | Dy547-labeled microRNA | 557/570 nm |
| Mimic Transfection | | mimic based on the | |
| Control with Dy547 | | C. elegans miRNA | |
| 5′ sense | | cel-miR-67 (miRIDIAN Mimic | |
| | | Negative Control #1) | |
| Custom miRIDIAN | 77C-CUSTOM- | SEQ ID NO: 27 Cy5-labeled | 645/665 nm |
| Mimic Transfection | NM-48 | microRNA mimic based | |
| Control with Cy5 | | on the C. elegans miRNA | |
| 5′ sense | | cel-miR-67 (miRIDIAN Mimic | |
| | | Negative Control #1) | |
| miRIDIAN | CP-301085-01 | AGGUGGUCCGUGGCGCGUUCGC | — |
| microRNA hsa- | | (MIMAT0004696) | |
| miR-323a-5p | | (SEQ ID NO: 1) | |
| mimic | | | |

(A/E, Absorbance/emission)

TABLE 2
siRNA

| Gene | | Sequence (5′ to 3′) |
|---|---|---|
| siControl 1 | siRNA Control | GUAAGACACGACUUAUCGC |
| | 1 | (SEQ ID NO: 25) |
| | siRNA Control | CAUUCUGUGCUGAAUAGCG |
| | 1_as | (SEQ ID NO: 26) |
| siCCND1 | CCND1 | CCUACGAUACGCUACUAUAUU |
| | | (SEQ ID NO: 12) |
| | CCND1_as | AAUAUAGUAGCGUAUCGUAGG |
| | | (SEQ ID NO: 13) |

(“as” means the antisense sequence; the antisense sequence: RNA that is perfectly complementary to the mRNA target sequence)

The siRNA were received from the supplier freeze-dried and they were resuspended in water for later use at the desired concentration.

### QS Synthesis

Quatsomes (QS) were composed of sterols, such as Chol, DC-Chol or Chems, and non-lipid cationic surfactants with high positive charge, such as MKC or CTAB. Different QS were prepared by tuning the ratio between Chol and modified sterols (DC-Chol or Chems):

QS₀: (0% Chol/100% Chems):MKC; QS₁: (100% Chol/0% DC-Chol):MKC; QS₂: (91% Chol/9% DC-Chol):MKC; QS₃: (53% Chol/47% DC-Chol):MKC; QS₄: (0% Chol/100% DC-Chol):MKC, QS₆: (90.5% Chol/9.5% DC-Chol):CTAB; QS₆: (51% Chol/49% DC-Chol):CTAB; QS₇: (0% Chol/100% DC-Chol):CTAB. All QS were prepared at molar ratio 1:1 between the different sterols and the surfactant (MKC or CTAB), except QS₁ which was prepared at 1:3 molar ratio. Moreover, QS₄ functionalized with Dil was prepared inserting Dil in the QS₄ membrane. Also, PEG-QS₄ were functionalized by PEG replacing some DC-Chol molecules achieving a final composition of QS₄: (10% Chol-PEG/90% DC-Chol):MKC at molar ratio 1:1.

QS were prepared using a methodology based on CF (Ferrer-Tasies et al. Langmuir. 2013 Jun. 4; 29(22):6519-28). Briefly, the sterols or derivatives thereof, such as Chol, Chems or DC-Chol, (see Table 3) were dissolved in EtOH (V_{EtOH}) at 313-318K for 10 minutes. Then, the organic phase was added to the vessel at working temperature (Tw=311K) and at atmospheric pressure. CO₂ was then added in order to obtain a volumetric expanded solution of the lipid at high pressure (Pw=11.5 MPa), 311 K and with a given CO₂ molar fraction (X_{CO2}=0.6). After 1 h of homogenization, this 002-expanded solution was depressurized, from working pressure (Pw) to atmospheric pressure, over an aqueous solution containing the non-lipid cationic surfactant (i.e. MKC or CTAB) (V_{H20}=8.3V_{EtOH}), see Table 3, to give uniform unilamellar nanovesicles in QS systems. This methodology can operate in a continuous mode or batch mode.

In order to prepare functionalized quatsomes, for example with fluorescent dyes, such as non-water soluble organic dyes, the method further comprised: the addition of Dil fluorophore (70 μM) in the organic phase formed by sterols and EtOH (V_{EtOH}), and then expanding the solution of the lipids with Dil by adding CO₂ at high pressure (Pw=11.5 MPa), 311 K and with a given CO₂ molar fraction (XCO₂=0.6). After 1 h of homogenization, this CO₂-expanded solution was depressurized, from working pressure (Pw) to atmospheric pressure, over an aqueous solution containing the non-lipid cationic surfactant (i.e. MKC or CTAB) (V_{H20}=8.3V_{EtOH}), see Table 3, to give uniform unilamellar fluorescent nanovesicles.

TABLE 3
Compositions used for the preparation of the various
QS systems by the DELOS-SUSP Method.

| | | | Membrane | % D-Chol*/ |
|---|---|---|---|---|
| | | | components | (Chol + |
| System | Organic phase | Aqueous phase | concentration | D-Chol) |
| QS₀ | Chems (0.069M) in EtOH | MKC (0.008M) in | 6.35 mg/mL | 100% |
| | | Water | | |
| QS₁ | Cholesterol (0.033M) in | MKC (0.011M) in | 5.00 mg/mL | 0% |
| | EtOH | Water | | |
| QS₂ | Cholesterol (0.066M) + | MKC (0.008M) | 5.70 mg/mL | 9% |
| | DC-Chol (0.006M) in EtOH | Water | | |
| QS₃ | Cholesterol (0.037M) + | MKC (0.008M) | 6.04 mg/mL | 47% |
| | DC-Chol (0.033M) in EtOH | Water | | |
| QS₄ | DC-Chol (0.065M) in EtOH | MKC (0.008M) | 6.46 mg/mL | 100% |
| | | Water | | |
| QS₄- | DC-Chol (0.065M) + 70 μM | MKC (0.008M) | 6.46 mg/mL | 100% |
| (Dil) | Dil in EtOH | Water | | |
| PEG- | DC-Chol (0.064M) + Chol- | MKC (0.008M) in | 7.1 mg/mL | 90% |
| QS₄ | PEG₁₀₀₀ (0.0069M) in etOH | Water | | |
| QS₅ | Cholesterol (0.006M) + | CTAB (0.008M) in | 5.7 mg/mL | 9.5% |
| | DC-Chol (0.007M) in EtOH | Water | | |
| QS₆ | Cholesterol (0.035M) + | CTAB (0.008M) in | 6.0 mg/mL | 49% |
| | DC-Chol (0.035M) in EtOH | Water | | |
| QS₇ | DC-Chol (0.069M) in EtOH | CTAB (0.008M) in | 6.5 mg/mL | 100% |
| | | Water | | |

*D-Chol means derived cholesterol, which can be DC-Chol or Chems

After one week of stabilisation, all samples were purified by diafiltration using the KrosFlo® Research Iii TFF System (Spectrum Labs from Repligen Corporation; Waltham, Mass., USA). Samples were diafiltered using a size-exclusion mPEs Micro Kros filter column (100 KDa molecular weight cut-off and a surface area of 20 cm²) to remove ethanol and the excess of material non-encapsulated in QS, thereby QS were finally in a MilliQ water media.

Nanovesicles comprising 100% DC-Chol as the sterol and MKC at a ratio 1:2 and 2:1 were also prepared (data not shown).

miRNA-QS Complexes Preparation:
a) Adding the corresponding volume in μL of QS (see table 7 for in vitro experiments and 11 for in vivo experiments) in a new eppendorf depending on the miRNA/QS loading desired.
b) Adding the corresponding volume in μL (see table 7 and 11 of miRNA above QS solution (depending on the desired final concentration of miRNA; i.e. 2.5 μL (stock concentration of 20 μM) to achieve a final concentration of miRNA of 2.5 μM for in vitro experiments and i.e. 42.6 μL of miRNA (stock concentration of 100 μM) to achieve a final concentration of miRNA of 21.3 μM for in vivo experiments).
c) Diluting the complexes in PBS 1× to ensure the mixing, avoid aggregation and maintained constant the miRNA concentration among the various QS-miRNA complexes.
d) After pipetting twice up-down (less than five minutes of incubation), complexes were formed.
e) When needed, adding the complexes formed to the cells.

### Physicochemical Characterisation

### Dynamic Light Scattering

Particle size, polydispersity and surface charge density of QS were evaluated using the dynamic light scattering (DLS) technique by Zetasizer Nano ZS (Malvern Instruments, Malvern, UK). The hydrodynamic diameter and polydispersity index (PDI) from three replicates of measurements were obtained using an incident He—Ne laser light of 4 mW, a wavelength of 633 nm and a detector angle fixed at 173° with homodyne detection. Samples were measured as born without modifications or dilution at 298K. Moreover, another non-invasive backscattering technique measured with Zetasizer Nano ZS was the Z-potential, which was determined at 298K in a DTS1070 disposable folded capillary cuvette. Values reported were the average of hydrodynamic diameters±standard deviation (SD) among samples or Z-potential±standard deviation (SD). Experiments were carried out at least in triplicate.

QS stability over time was determined by DLS after one week, two weeks, one month, three months, six months and one year after sample preparation or purification. QS were considered stable over time when until two months the hydrodynamic diameter remained smaller than 300 nm and the PDI remained in the range between 0.1-0.3.

### Cryo-TEM

CryoTEM images were acquired with a JEOL JEM 2011 transmission electron microscope (JEOL, Tokyo, Japan) at 200 KV. Samples were placed on a Holey carbon grid or a copper grid coated with perforated polymer film before being frozen in liquid ethane. The Gatan 626 cryo-transfer system was inserted into the microscope. Images were recorded using a Gatan Ultrascan US1000 CCD camera and analysed with the Digital Micrograph 1.8 program.

### pH Buffering Capacity

The buffer capacity of QS was determinated by acid-base titration. Briefly, QS were at a final concentration of 5 mg/mL in aqueous solution. The resulting solution was adjusted to pH 9 with sodium hydroxide (0.01M). The titration curve was determined by stepwise addition of 10 μL aliquots of hydrochloric acid (0.01 M). The pH was measured after each addition with a pH meter (Hanna Instruments, Woonsocket, R.I., USA) until pH 2 was reached.

### Results:

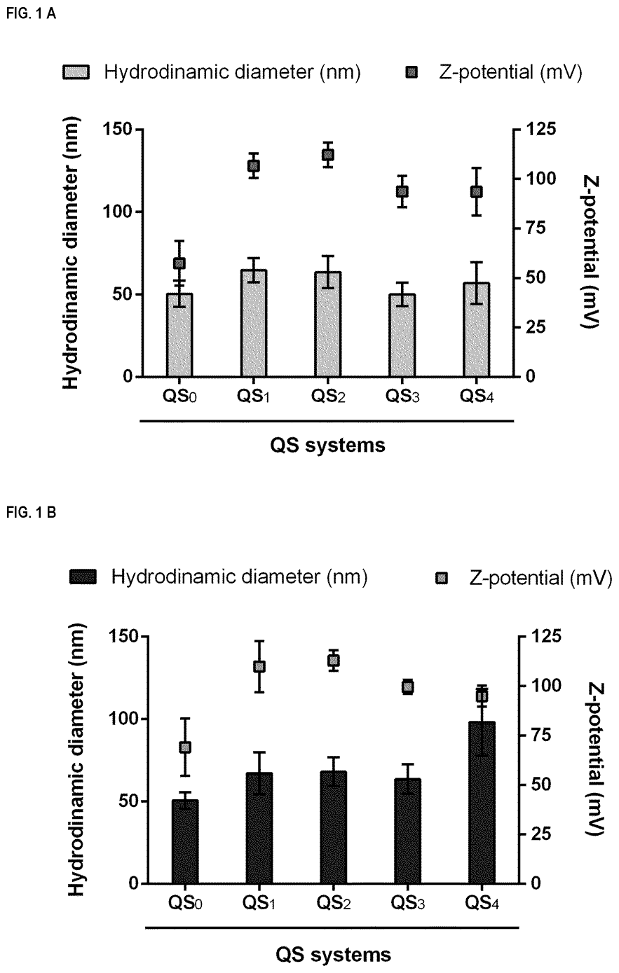

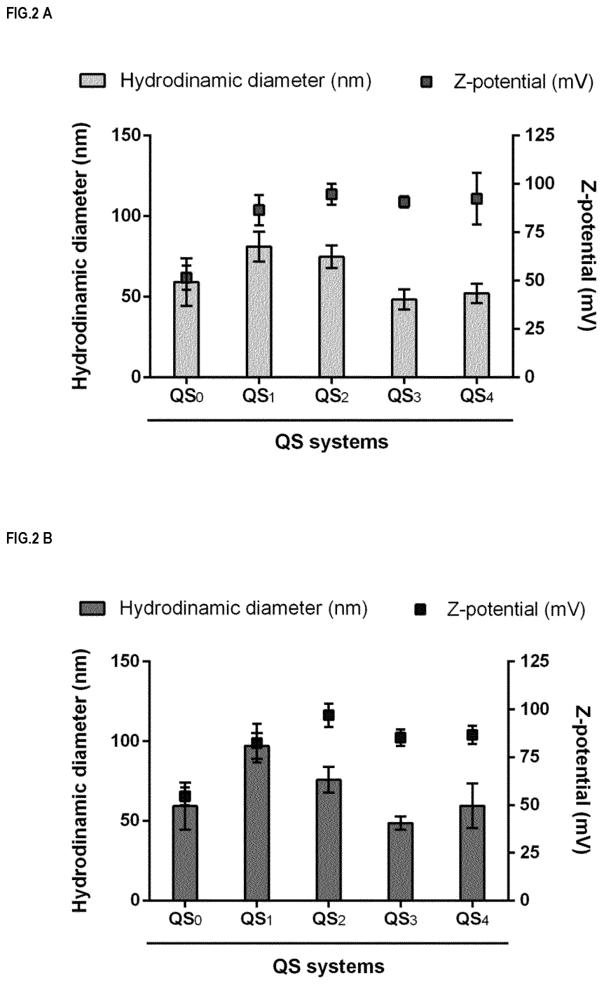

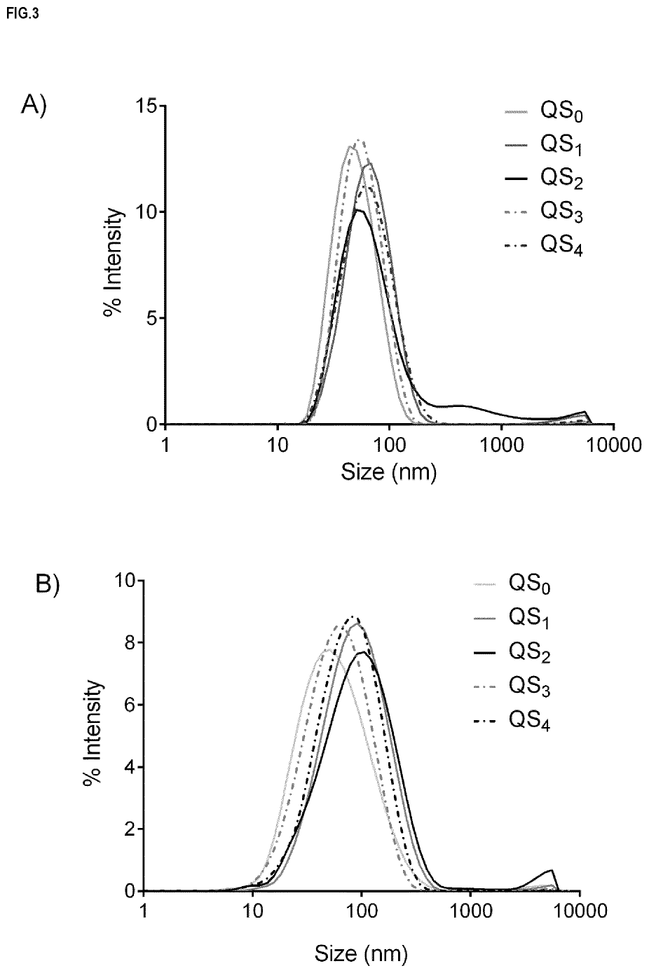

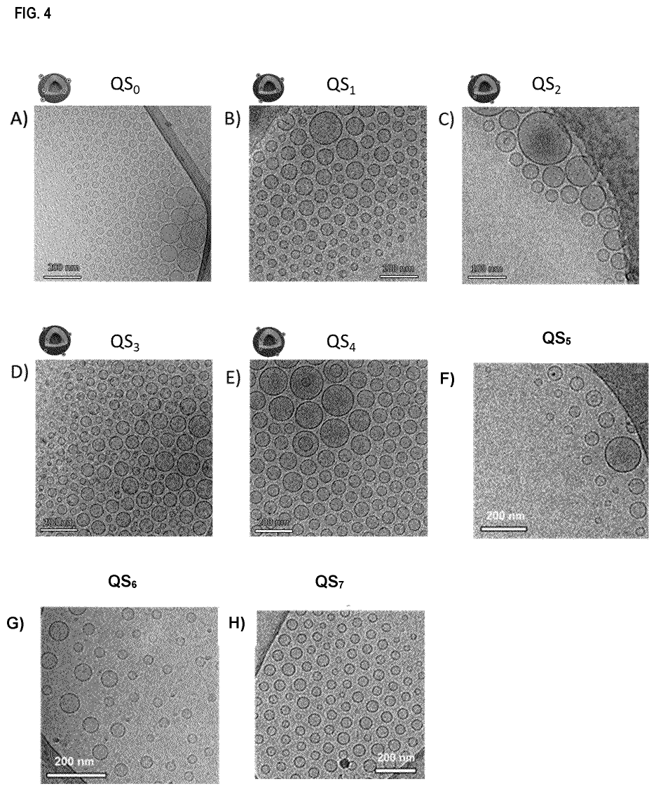

QS were prepared varying the sterols and surfactants composition in all cases (QS₀: (0% Chol/100% Chems):MKC; QS_{T}: (100% Chol/0% DC-Chol):MKC; QS₂: (91% Chol/9% DC-Chol):MKC; QS₃: (53% Chol/47% DC-Chol):MKC; QS₄: (0% Chol/100% DC-Chol):MKC), QS₅: (90.5% Chol/9.5% DC-Chol):CTAB; QS₆: (51% Chol/49% DC-Chol):CTAB; QS₇: (0% Chol/100% DC-Chol):CTAB. DLS measurements (see Table 4) and cryo-TEM images (FIG. 4) revealed that all the obtained quatsomes (QS) were nanovesicles with homogeneous size, spherically-shaped and unilamellar. DLS measurements (see FIG. 3) revealed an unimodal size distribution centred in an average size around 100 nm. Added to that, QS presented low polydispersity and high colloidal stability over time since minor variations in the size distributions were found for all QS (FIGS. 1 and 2). Positive charges from QS guaranteed a high complexation efficiency with negative charges of small RNA (sRNA) by electrostatic interactions.

TABLE 4
Physicochemical properties of QS systems after DELOS-
SUSP preparation and after purification by diafiltration.

| | Hydrodynamic | | |
|---|---|---|---|
| | diameter | Polidispersity | Z-potential |
| | (nm) | index | (mV) |
| QS systems | | | |
| after | | | |
| preparation | | | |
| QS₀ | 50.5 ± 8.0 | 0.28 ± 0.12 | 52.8 ± 5.4 |
| QS₁ | 64.8 ± 7.4 | 0.24 ± 0.02 | 106.7 ± 6.2 |
| QS₂ | 63.5 ± 9.8 | 0.39 ± 0.14 | 112.2 ± 6.2 |
| QS₃ | 50.1 ± 7.1 | 0.15 ± 0.01 | 93.7 ± 7.9 |
| QS₄ | 51.1 ± 7.8 | 0.17 ± 0.03 | 92.8 ± 8.6 |
| ^{Dil}QS₄ | 45.2 ± 3.5 | 0.23 ± 0.02 | 48.8 ± 7.0 |
| PEG-QS₄ | 49.0 ± 2.0 | 0.15 ± 0.01 | 82.5 ± 6.4 |
| QS₅ | 70.1 ± 4.2 | 0.34 ± 0.08 | 114.7 ± 4.0 |
| QS₆ | 74.6 ± 5.0 | 0.16 ± 0.03 | 93.5 ± 9.6 |
| QS₇ | 78.3 ± 7.8 | 0.18 ± 0.00 | 99.0 ± 4.24 |
| QS systems | | | |
| after | | | |
| purification | | | |
| QS₀ | 59.2 ± 8.8 | 0.40 ± 0.07 | 51.6 ± 6.2 |
| QS₁ | 73.5 ± 10.8 | 0.26 ± 0.02 | 91.3 ± 13.1 |
| QS₂ | 74.8 ± 7.0 | 0.44 ± 0.05 | 94.7 ± 5.4 |
| QS₃ | 43.4 ± 6.3 | 0.27 ± 0.02 | 90.8 ± 2.9 |
| QS₄ | 52.2 ± 6.0 | 0.29 ± 0.04 | 86.7 ± 4.7 |
| ^{Dil}QS₄ | 62.3 ± 6.5 | 0.25 ± 0.02 | 63.1 ± 4.9 |
| PEG-QS₄ | 62.0 ± 1.0 | 0.28 ± 0.02 | 77.0 ± 2.0 |
| QS₅ | 72.5 ± 3.1 | 0.27 ± 0.01 | 94.8 ± 5.0 |
| QS₆ | 78.0 ± 4.4 | 0.15 ± 0.00 | 76.5 ± 7.4 |
| QS₇ | 64.0 ± 4.7 | 0.17 ± 0.06 | 83.6 ± 1.3 |

Moreover, QS with a high presence of DC-Chol in their compositions presented a pH sensitive behaviour, maintaining constant the pH in acidic conditions. QS₄ ((0% Chol/100% DC-Chol):MKC) showed the best buffering capacity (FIG. 5).

### 2. Colloidal Structures

### 2.1—Colloidal Structures Comprising Cholesterol, Chol-VS and CTAB in Aqueous Medium

### Materials and Methods:

Cholesten-3β-ol (Choi, purity 95%; #A0807) was obtained from PanReac (Castellar del Valles, Spain). Cetyltrimethylammonium bromide (CTAB, ultra for molecular biology) was purchased from Fluka-Aldrich. Cholest-5-ene, 3[2-(ethenylsulfonyl)ethoxy]-,(3b)-(Chol-VS) was synthetized and characterized. Ethanol was purchased from Teknochroma (Sant Cugat del Valles, Spain). Carbon dioxide (purity 99.9%) was acquired from Carburos Metálicos S. A. (Cornelia de Llobregat, Spain). All the chemicals were used without further purification and all solutions were prepared using pre-treated Milli-Q water (Millipore Ibérica, Madrid, Spain).

Synthesis of cholest-5-ene, 3-[2-(ethenylsulfonyl)ethoxy]-, (3β)-(Chol-VS)

To a solution of cholesterol (300 mg, 0.77 mmol) in THF (20 ml) was added divinyl sulfone (0.12 ml, 1.16 mmol) and potassium tert-butoxide (9 mg, 0.077 mmol). The reaction mixture was magnetically stirred at room temperature for 1 h. Amberlita IR 120H was then added and the magnetic stirring continued for additional 30 min. After filtration, the solvent was evaporated under reduced pressure. TLC of the crude showed the presence of cholesterol. Acetic anhydride (8 ml) and pyridine (4 ml) were added to the resulting crude and the new reaction mixture was kept at room temperature for 16 h. Acetylation of the crude reaction allowed the separation of compound Chol-VS. Evaporation under reduced pressure gave a crude that was purified by column chromatography (ether:hexane 1:2) yielding compound Chol-VS as a solid (204 mg, 52%).

M.P. 133 −135° C.; [α]_{D}−19 (c 1, chloroform); vₘₐₓ(KBr)/cm⁻¹: 3409, 1461, 1373, 1319, 1115, and 1052; ¹H-NMR (CDCl₃, 400 MHz): δ 6.75 (dd, 1H, J=16.7 and 9.9 Hz), 6.40 (d, 1H, J=16.7 Hz), 6.07 (d, 1H, J=9.9 Hz), 5.35 (br s, 1H), 3.88 (t, 2H, J=5.6 Hz), 3.23 (t, 2H, J=5.6 Hz), 3.19 (m, 1H), 2.35-1.84 (several m, 7 H), 1.56-0.95 (several m, 21H), 0.99 (s, 3H), 0.92 (d, 3H, J=6.4 Hz), 0.86 (d, 6H, J=6.6 Hz), 0.67 (s, 3H); ¹³C-NMR (CDCl3, 125 MHz): δ 140.2, 138.0, 128.5, 122.1, 79.8, 61.5, 56.7, 56.1, 55.4, 50.1, 42.3, 39.7, 39.5, 38.8, 37.0, 36.8, 36.2, 35.8, 31.9, 31.8, 28.2, 28.1, 28.0, 24.3, 23.8, 22.9, 22.5, 21.0, 19.3, 18.7, 11.8; HRMS (m/z) (FAB+) calcd. for C₃₁H₅₂O₃SNa [M+Na]⁺: 527.3535; found: 527.3535.

### Synthesis of Colloidal Structures

Colloidal structures (CS) were composed of sterols, such as Chol, Chol-VS, and quaternary ammonium surfactants with high positive charge, such as CTAB at molar ratio 1:1 between the sterols (Chol+Chol-VS) and the CTAB surfactant.

Colloidal structures were prepared using the methodology previously described (Ferrer-Tasies et al. Langmuir. 2013 Jun. 4; 29(22):6519-28). Briefly, the sterols or derivatives thereof, such as Chol, Chol-VS, (see Table 5) were dissolved in EtOH at 308K. Then, the organic phase was added to the vessel at working temperature (Tw=308K) and at atmospheric pressure. CO₂ was then added in order to obtain a volumetric expanded solution of the lipid at high pressure (Pw=10 MPa), 311 K and with a given CO₂ molar fraction (XCO₂=0.8). After 1 h of homogenization, this CO₂-expanded solution was depressurized, from working pressure (Pw) to atmospheric pressure, over a continuous aqueous flow containing the non-lipid cationic surfactant CTAB (see Table 5), to give different colloidal structures depending on the ratio between Chol and Chol-VS.

TABLE 5
Compositions Used for the Preparation of the various
colloidal structures (CS) by the DELOS-SUSP Method.

| | | | Membrane | % Chol-VS/ |
|---|---|---|---|---|
| | | | components | Chol |
| System | Organic phase | Aqueous phase | concentration | (Total) |
| QS | Chol (0.032M) in EtOH | CTAB (0.008M) in | 4.83 mg/mL | 0% |
| | | Water | | |
| CS-VS₁ | Cholesterol (0.022M) + | CTAB (0.008M) in | 5.07 mg/mL | 32% |
| | Chol-VS (0.010M) in EtOH | Water | | |
| CS-VS₂ | Cholesterol (0.016M) + | CTAB (0.008M) in | 5.20 mg/mL | 49% |
| | Chol-VS (0.016M) in EtOH | Water | | |
| CS-VS₃ | Cholesterol (0.011M) + | CTAB (0.008M) in | 5.33 mg/mL | 66% |
| | Chol-VS (0.021M) in EtOH | Water | | |
| CS-VS₄ | Cholesterol (0.008M) + | CTAB (0.008M) in | 5.39 mg/mL | 74% |
| | Chol-VS (0.024M) in EtOH | Water | | |
| CS-VS₅ | Chol-VS (0.032M) in EtOH | CTAB (0.008M) in | 5.59 mg/mL | 100% |
| | | Water | | |

### Results:

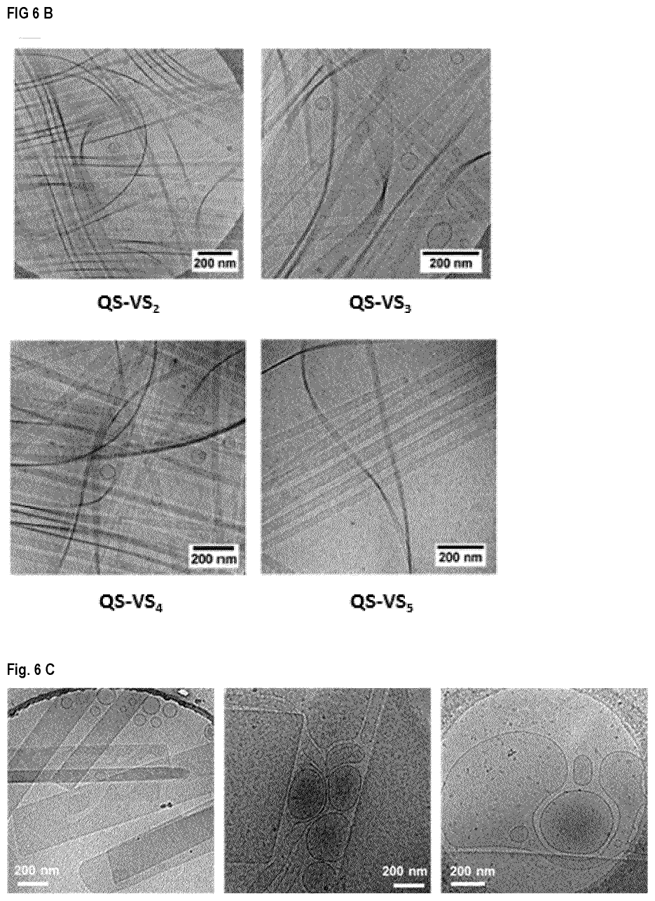

CS-VS were prepared varying the sterols and surfactants composition in all cases (CS-VS₀: (0% Chol-VS/100% Chol):CTAB; CS-VS1: (32% Chol-VS/68% Chol):CTAB; CS-VS2: (49% Chol-VS/51% Chol):CTAB; CS-VS₃: (66% Chol-VS/34% Chol):CTAB; CS-VS₄: (74% Chol-VS/26% Chol):CTAB; CS-VS₅ (100% Chol-VS/0% Chol):CTAB. DLS measurements (see Table 6) and cryo-TEM images (see FIG. 6) revealed that self-assembling of the progressive substitution of the cholesterol molecule, in an equimolar mixture Chol:CTAB, by novel cholesterol molecules bearing vinyl sulphone (Chol-VS), leaded to a different colloidal self-assembly behavior.

TABLE 6
Physicochemical properties of CS systems after
DELOS-SUSP preparation.

| | | Hydrodynamic diameter |
|---|---|---|
| | Morphological | (D) in nm & Polidispersity |
| Systems | description | index (Pdl) |
| QS | Nanovesicles | D = 61.96 ± 0.72; |
| | | Pdl = 0.16 ± 0.004 |
| CS-VS₁ | Nanovesicles & | D = 73.84 ± 0.53; |
| | Nanoribbons | Pdl = 0.150 ± 0.005 |
| CS-VS₂ | Mainly Nanoribbons | Submicron range^{a} (>10 μm) |
| CS-VS₃ | Mainly Nanoribbons | Submicron range^{a} (>10 μm) |
| CS-VS₄ | Mainly Nanoribbons | Submicron range^{a} (>10 μm) |
| CS-VS₅ | Nanoribbons | Submicron range^{a} (>10 μm) |

^{a}Sample size exceeds the measuring range.

On one hand, DLS measurements of QS-VS systems from QS-VS₂ to QS-VS₄ did not meet quality criteria because of presence of large structures. Besides, all cryo-TEM images exhibited the coexistence of mainly thin micrometer-long ribbons (nanoribbons) and few unilamellar spherical. On the other hand, cryo-TEM images in the case of the complete substitution of Chol by Chol-VS, CS-VS₅ system, vesicle-like assemblies were not formed, and only nanoribbons assemblies were found.

### 2.2—Colloidal Structures Comprising Cholesterol and MKC

This system was composed of 100% Chol/0% DC-Chol:MKC and was prepared at molar ratio 1:1 between sterol and the surfactant in milliQ pure water with 10% of EtOH.

CS-CH were prepared using a methodology based on CF (Ferrer-Tasies et al. Langmuir. 2013 Jun. 4; 29(22):6519-28). Briefly, the sterol (Chol) was dissolved in EtOH (V_{EtOH}) at 313-318K for 10 minutes. Then, the organic phase was added to the vessel at working temperature (Tw=311K) and at atmospheric pressure. CO₂ was then added in order to obtain a volumetric expanded solution of the lipid at high pressure (Pw=11.5 MPa), 311 K and with a given CO₂ molar fraction (X_{CO2}=0.6). After 1 h of homogenization, this CO₂-expanded solution was depressurized, from working pressure (Pw) to atmospheric pressure, over an aqueous solution containing the non-lipid cationic surfactant (i.e. MKC) (V_{H20}=8.3V_{EtOH}), to give colloidal structures. This methodology operated in a continuous mode or batch mode. Compositions used for the preparation of the CS-CH system by the DELOS-SUSP Method. The compositions used for the preparation of the CS-CH system had as organic phase Chol (0.070M) in EtOH; as aqueous phase, MKC (0.008M) in water; the membrane components concentration was 5.6 mg/mL and the % D-Chol/(Chol+D-Chol) was of 0%.

### Results:

The system CS-CH did not form nanovesicles. DLS measurements and cryo-TEM images (see FIG. 6C) revealed that self-assembling of the cholesterol molecule with MKC, in an equimolar mixture, leaded to a different colloidal self-assembly behavior forming preferably nanoribbons. DLS measurements of CS-CH systems did not meet quality criteria because of presence of large structures: Hydrodynamic diameter (D) in nm, D=174.3±33.2; Polidispersity index (PdI)=0.56±0.09. Besides, all cryo-TEM images exhibited the coexistence of mainly thin micrometer-long ribbons (nanoribbons) and few unilamellar spherical.

### Example 3: QS-sRNA Complex Formation

### Materials and Methods:

QS-sRNA complexes were formulated by mixing QS and small RNA (sRNA) at different sRNA-to-QS mass ratios (w/w) called QS-sRNA loadings. First of all, for in vitro experiments, QS were diluted in Depc treated water (ThermoFisher; #750024) to achieve the desired concentrations, such as 3.98 mg/mL for QS₀; 1.15 mg/mL for QS₁; 1.76 mg/mL for QS₂; 1.88 mg/mL for QS₃ and 1.99 mg/mL for QS₄. To form QS-sRNA complexes, 2.5 μL of sRNA were added over the appropriate volume (μL) of QS solution to obtain the desired sRNA-to-QS mass ratios (w/w), and maintaining a constant sRNA concentration (see Table 7). To achieve a constant final concentration of sRNA (2.5 μM), QS-sRNA complexes were diluted with PBS 1× until reach the desired final volume (i.e. 20 μL), then mixed by pipetting twice up-down (less than five minutes of incubation). The resulting QS-sRNA complexes were generated by ionic interactions between the positive charges on the surface of QS and the negative charges of sRNA. The different sRNA-to-QS mass ratios (w/w) were calculated between the QS mass and sRNA mass, depending on QS composition.

TABLE 7
Loadings of QS-sRNA complexes prepared at different sRNA-to-QS mass ratios (to reach a final
volume of i.e. 20 μL for in vitro experiments). sRNA used were miRNA or siRNA.

| QS₀-sRNA | [QS₀ | | [sRNA | | Mass ratio | Mass ratio |
|---|---|---|---|---|---|---|
| complexes | stock] | Volume of | stock] | Volume of | miRNA/QS₀ · | siRNA/QS₀ · |
| (loadings) | (mg/mL) | QS (μL) | (μM) | sRNA (μL) | 10⁻² | 10⁻² |
| QS₀-sRNA (I) | 3.98 | 8.75 | 20 | 2.5 | 2.02 | 1.91 |
| QS₀-sRNA (II) | | 6.56 | | | 2.70 | 2.55 |
| QS₀-sRNA (III) | | 4.375 | | | 4.05 | 3.82 |
| QS₀-sRNA (IV) | | 2.625 | | | 6.75 | 6.37 |
| QS₀-sRNA (V) | | 1.58 | | | 11.21 | 10.58 |
| QS₀-sRNA (VI) | | 1.05 | | | 16.87 | 15.91 |
| QS₀-sRNA (VII) | | 0.7 | | | 25.31 | 23.87 |
| QS₀-sRNA(VIII) | | 0.42 | | | 42.18 | 39.78 |
| QS₁-sRNA | [QS₁ | | [sRNA | | Mass ratio | Mass ratio |
| complexes | stock] | Volume of | stock] | Volume of | miRNA/QS₁ · | siRNA/QS₁ · |
| (loadings) | (mg/mL) | QS (μL) | (μM) | sRNA (μL) | 10⁻² | 10⁻² |
| QS₁-sRNA (I) | 1.15 | 17.5 | 20 | 2.5 | 3.10 | 2.92 |
| QS₁-sRNA (II) | | 13.13 | | | 4.13 | 3.90 |
| QS₁-sRNA (II) | | 8.75 | | | 6.20 | 5.85 |
| QS₁-sRNA (IV) | | 4.38 | | | 12.40 | 11.69 |
| QS₁-sRNA (V) | | 2.63 | | | 20.66 | 19.49 |
| QS₁-sRNA (VI) | | 1.75 | | | 30.99 | 29.23 |
| QS₁-sRNA (VII) | | 1.17 | | | 46.35 | 43.72 |
| QS₁-sRNA (VIII) | | 0.7 | | | 77.47 | 73.08 |
| QS₂-sRNA | [QS₂ | | [sRNA | | Mass ratio | Mass ratio |
| complexes | stock] | Volume of | stock] | Volume of | miRNA/QS₂ · | siRNA/QS₂ · |
| (loadings) | (mg/mL) | QS (μL) | (μM) | sRNA (μL) | 10⁻² | 10⁻² |
| QS₂-sRNA (I) | 1.76 | 17.5 | 20 | 2.5 | 2.29 | 2.16 |
| QS₂-sRNA (II) | | 13.13 | | | 3.05 | 2.88 |
| QS₂-sRNA (III) | | 8.75 | | | 4.58 | 4.32 |
| QS₂-sRNA (IV) | | 4.38 | | | 9.16 | 8.64 |
| QS₂-sRNA (V) | | 2.63 | | | 15.26 | 14.39 |
| QS₂-sRNA (VI) | | 1.75 | | | 22.89 | 21.59 |
| QS₂-sRNA (VII) | | 1.17 | | | 34.24 | 32.29 |
| QS₂-sRNA (VIII) | | 0.7 | | | 57.22 | 53.98 |
| QS₃-sRNA | [QS₃ | | [sRNA | | Mass ratio | Mass ratio |
| complexes | stock] | Volume of | stock] | Volume of | miRNA/QS₃ · | siRNA/QS₃ · |
| (loadings) | (mg/mL) | QS (μL) | (μM) | sRNA (μL) | 10⁻² | 10⁻² |
| QS₃-sRNA (I) | 1.88 | 17.5 | 20 | 2.5 | 2.14 | 2.02 |
| QS₃-sRNA (II) | | 13.13 | | | 2.86 | 2.70 |
| QS₃-sRNA (III) | | 8.75 | | | 4.29 | 4.04 |
| QS₃-sRNA (IV) | | 4.38 | | | 8.57 | 8.09 |
| QS₃-sRNA (V) | | 2.63 | | | 14.29 | 13.48 |
| QS₃-sRNA (VI) | | 1.75 | | | 21.43 | 20.21 |
| QS₃-sRNA (VII) | | 1.17 | | | 32.05 | 30.23 |
| QS₃-sRNA (VIII) | | 0.7 | | | 53.57 | 50.53 |
| QS₄-sRNA | [QS₄ | | [sRNA | | Mass ratio | Mass ratio |
| complexes | stock] | Volume of | stock] | Volume of | miRNA/QS₄ · | siRNA/QS₄ · |
| (loadings) | (mg/mL) | QS (μL) | (μM) | sRNA (μL) | 10⁻² | 10⁻² |
| QS₄-sRNA (I) | 1.99 | 17.5 | 20 | 2.5 | 2.02 | 1.91 |
| QS₄-sRNA (II) | | 13.13 | | | 2.70 | 2.55 |
| QS₄-sRNA (III) | | 8.75 | | | 4.05 | 3.82 |
| QS₄-sRNA (IV) | | 4.38 | | | 8.10 | 7.64 |
| QS₄-sRNA (V) | | 2.63 | | | 13.50 | 12.73 |
| QS₄-sRNA (VI) | | 1.75 | | | 20.24 | 19.10 |
| QS₄-sRNA (VII) | | 1.17 | | | 30.28 | 28.56 |
| QS₄-sRNA (VIII) | | 0.7 | | | 50.61 | 47.74 |

### Gel Electrophoresis:

Agarose electrophoresis gels were prepared at 2.5% of agarose in Tris/Acetate/EDTA (TAE 1×) and 0.005% of Ethidium Bromide. QS-sRNA complexes were prepared and, after five minutes of incubation, complexes were loaded in each well of the gel in PBS loading buffer (2.5% of glycerol).

To separate miRNA from QS 0.25% of SDS was added in specified wells. Gels were run at 120V for one hour. Electrophoresis images were acquired using the Gel Doc XR+System (Biorad, Hercules, Calif., USA).

### Results:

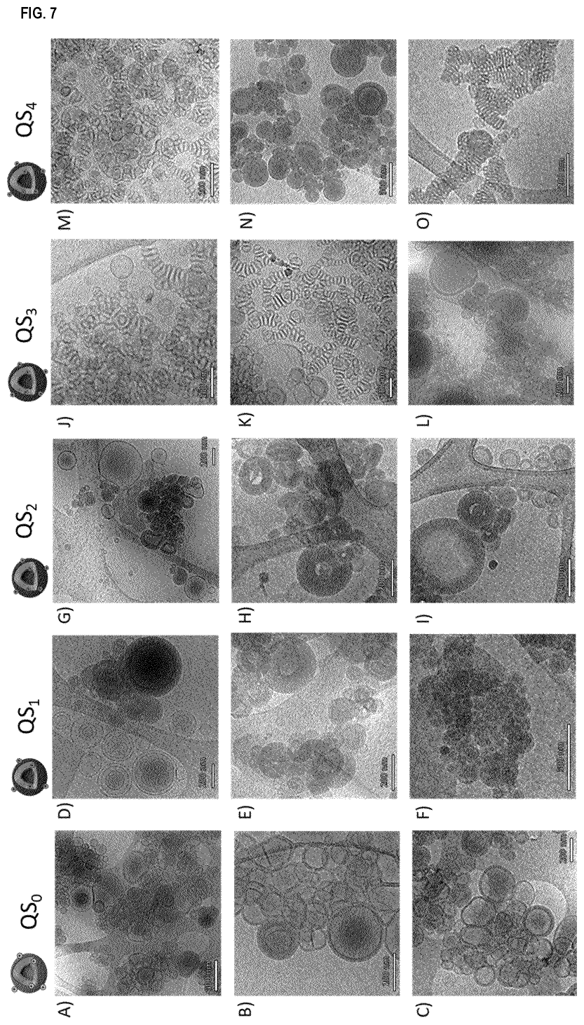

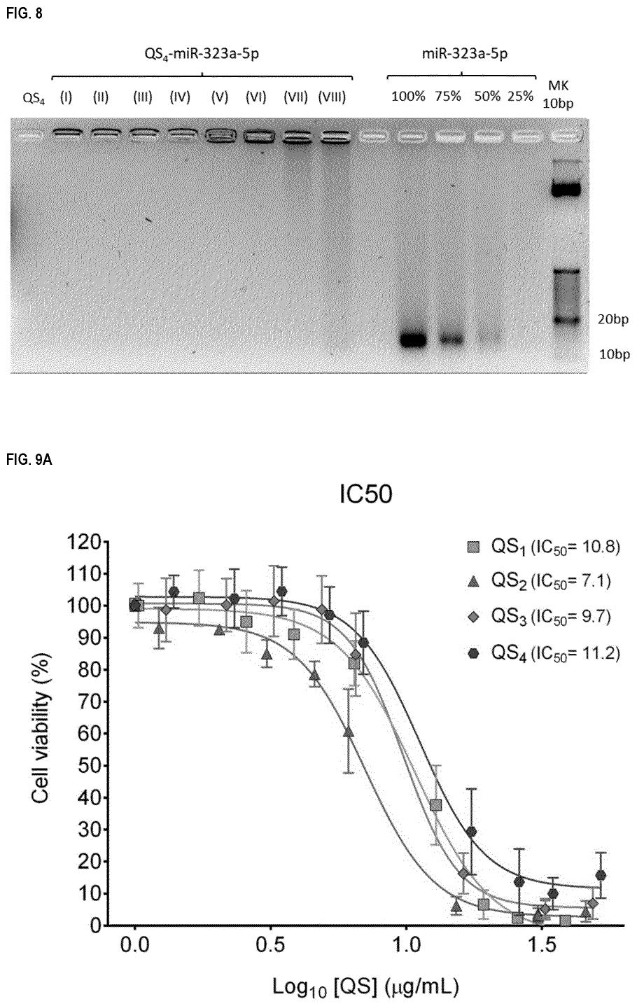

QS-miRNA complexes presented different morphology depending on QS composition and the ratio in mass between QS and miRNA. QS₀₋₂ presented more multilayer structures than QS₃₋₄, which present bunch structures; on the other hand, high miRNA-to-QS mass ratios present bigger aggregates than low miRNA-to-QS mass ratios (see FIG. 7).

QS₀ could not complex the miRNA with a 100% of efficiency even at low loadings of miRNA- per -QS (such as (I and II). Complexation efficiency was directly proportional to increasing DC-Chol compositions in QS. So, QS₁ (which 0% of DC-Chol) presented less complexation efficiency than QS₂₋₄. However, QS₂₋₄ already presented a 100% of complexation at loading QS-miRNA (VI), while with QS₁ the fully complexation was at loading QS-miRNA (IV).

### Example 4: Cell Viability Study

### Materials and Methods:

### Cell Cultures

SK-N-BE(2) were acquired from Public Health England Culture Collections (Salisbury, UK) and stored in liquid nitrogen. Upon resuscitation, SK-N-BE(2) cells were cultured in Iscove's modified Dulbecco's Medium (Life Technologies, Thermo Fisher Scientific, Waltham, Mass., USA), supplemented with 10% heat-inactivated foetal bovine serum (FBS) South America Premium, 1% of Insulin-Transferrin-Selenium Supplement (Life Technologies, Thermo Fisher Scientific), 100 U/mL penicillin, 100 μg/mL streptomycin (Life Technologies, Thermo Fisher Scientific) and 5 μg/mL plasmocin (InvivoGen, San Diego, Calif., USA). All cultures were maintained at 37° C. in a saturated atmosphere of 95% air and 5% CO₂. SK-N-BE(2) cells were tested for mycoplasma contamination periodically.

For in vitro experiments, SK-N-BE(2) neuroblastoma cells were reverse transfected with the addition of the QS-sRNA complexes to complete cell culture medium (IMDM, 10% heat-inactivated foetal bovine serum (FBS) South America Premium (Biowest, Nuaillé, France)) without antibiotics. After overnight (o/n) incubation media was changed for IMDM supplemented with 10% FBS and antibiotics.

### Cell Viability Assays

To test QS or QS-miRNA complexes cytotoxicity, SK-N-BE(2) cells were seeded in 96-well plates at 18×103 cells/well (6 replicates/condition) and treated with QS (0.7 μg/mL to 52 μg/mL; Table 8) or reverse transfected with 2.5 μM of miR-Control Dy547 (which has the same sequence as the el microRNA control 1 disclosed in table 1; Dharmacon Inc (Lafayette, Colo., USA) complexed with QS at various miRNA-to-QS mass ratios (loadings of QS-miRNA) (I to VIII), to achieve a final miRNA concentration of 50 nM.

TABLE 8
Formulations used for Cell Viability Assays

| Mass ratio | | | | | |
|---|---|---|---|---|---|
| miRNA/QS | | | | | |
| (loadings of QS- | [miRNA] | [c] QS₁ | [c] QS₂ | [c] QS₃ | [c] QS₄ |
| miRNA) | (nM) | (μg/mL) | (μg/mL) | (μg/mL) | (μg/mL) |
| QS-miRNA (I) | 50 | 25.73 | 30.61 | 32.52 | 34.76 |
| QS-miRNA (II) | | 19.29 | 22.95 | 24.39 | 26.07 |
| QS-miRNA (III) | | 12.86 | 15.30 | 16.26 | 17.38 |
| QS-miRNA (IV) | | 6.43 | 6.12 | 8.13 | 8.69 |
| QS-miRNA (V) | | 3.86 | 4.59 | 4.88 | 5.21 |
| QS-miRNA (VI) | | 2.57 | 3.06 | 3.25 | 3.48 |
| QS-miRNA (VII) | | 1.72 | 2.05 | 2.17 | 2.32 |
| QS-miRNA (VIII) | | 1.02 | 1.22 | 1.30 | 1.39 |

### Results:

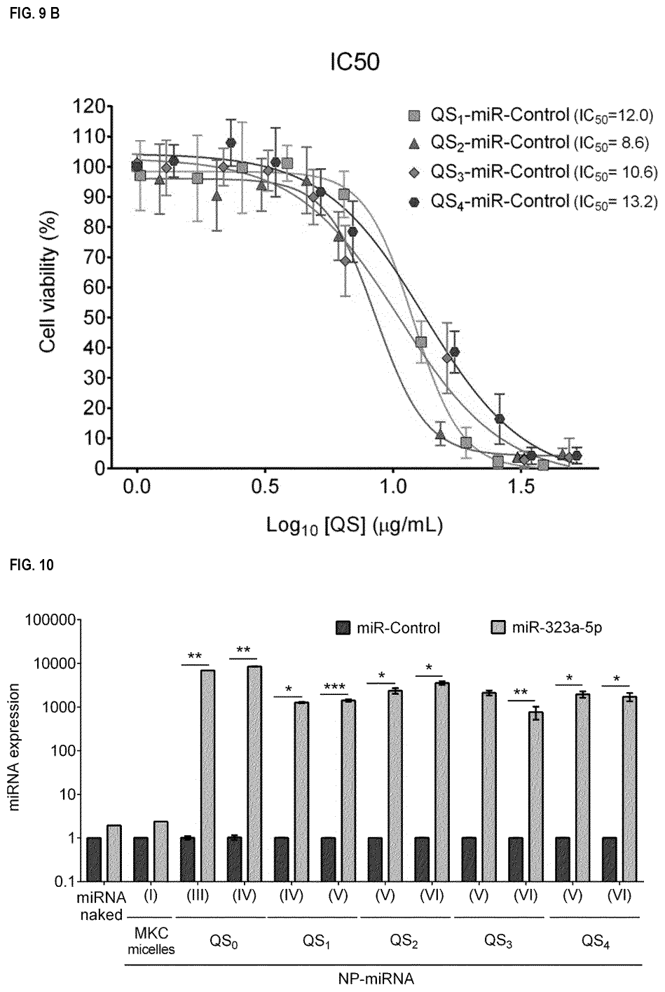

QS₁₋₄ presented a high viability (80-90%), even in low miRNA-to-QS mass ratio (loading of QS-miRNA) such as QS-miRNA (III). QS₁₋₄-miRNA presented higher viability than QS₁₋₄ not complexed owing to the shielding of positive charges from QS with miRNA negative charges (FIG. 9).

### Example 5: miRNA and siRNA Expression Using QS and Functionalization of QS

### Materials and Methods:

For QS-sRNA complexes efficacy assay, SK-N-BE(2) cells were seeded in 96-well plates at 9×10³ cells/well (6 replicates/condition) and reverse transfected with 50 nM of sRNA final concentration. The QS-sRNA complexes were formed as described in example 1. Twenty-four or ninety-six hours post-transfection, respectively, cells were fixed with 1% glutaraldehyde (Sigma-Aldrich) and stained with 0.5% crystal violet (Sigma-Aldrich). Crystals were dissolved in 15% acetic acid (Fisher Scientific, Hampton, Nou Hampshire, USA) and absorbance was measured at 590 nm using an Epoch Microplate Spectrophotometer (Biotek, Winooski, Vt., USA). The effect of QS-sRNA complexes on cell viability was normalized to mock-control-transfected cells.

Quantitative Real-Time PCR (qPCR)

Total RNA including small RNAs was extracted using the miRNeasy Mini Kit (Qiagen, Las Matas, Spain). mRNAs were reverse transcribed (0.5 μg total RNA) using Taqman RT kit (#4366596; Applied Biosystems, Thermo Fisher Scientific), and mature miRNA expression analysis was quantified using Taqman microRNA assays (#4440047; Applied Biosystems, Thermo Fisher Scientific) following manufacturer's recommendations. cDNA was quantified by standard RT-qPCR methodology using 2× Power SYBR Green Master Mix (Applied Biosystems, Thermo Fisher Scientific) using the ABI700SDS equipment. Gene expression was normalized against the L27 housekeeping gene for mRNA, and RNU-44 small RNA for miRNA analysis (#4427975). The primer sequences are listed in Table 9 and 10, respectively. The relative fold-change Relative quantification of gene expression was performed with a comparative 2^{(-ΔΔCT)} method (Livak K J, Schmittgen T D. Analysis of relative gene expression data using real-time quantitative PCR and the 2(−Delta Delta C(T)) method. Methods 2001; 25: 402-408).

TABLE 9
qPCR primers sequence list:

| | | Ampli- |
|---|---|---|
| | | con |
| Gene | Primer sequence (5′ to 3′) | (bp) |
| CHAF1A | Fw: TCA CCC AAT TCA TGA AGA AGC | 113 |
| | (SEQ ID NO: 28) | |
| | Rv: GAT CAT ACA GTC GCC CTC CT | |
| | (SEQ ID NO: 29) | |
| CCND1 | Fw: GCT GCG AAG TGG AAA CCA TC | 135 |
| | (SEQ ID NO: 30) | |
| | Rv: CCT CCT TCT GCA CAC ATT TGA A | |
| | (SEQ ID NO: 31) | |
| L27 | Fw: AGC TGT CAT CGT GAA GAA | 88 |
| | (SEQ ID NO: 32) | |
| | Rv: CTT GGC GAT CTT CTT CTT GCC | |
| | (SEQ ID NO: 33) | |

TABLE 10
qPCR primers sequence list:

| | | TaqMan Assay |
|---|---|---|
| miRBase | | ID and miRBase |
| ID | Mature miRNA Sequence | Accession No |
| hsa-miR- | AGGUGGUCCGUGGCGCGUUCGC | 002695 |
| 323a-5p | (SEQ ID NO: 1) | (MI0000807) |
| RNU44 | CCTGGATGATGATAGCAAATGC | 001094 |
| | TGACTGAACATGAAGGTCTT | (NR_002750) |
| | (SEQ ID NO: 34) | |

The RNU44 is as a housekeeping gene, commonly used to normalize the smallRNA content in the analyzed samples. In the qPCR performed for the hsa-miR-323a-5p and RNU44, according to manufactures instructions, TaqMan MicroRNA Assays employed a target-specific stem-loop primer, for the hsa-miR-323a-5p and RNU44, during cDNA synthesis to produce a template for real-time PCR).

### Western Blot

Protein extracts were obtained in RIPA buffer 1× (ThermoFisher Scientific), supplemented with 1×EDTA-free complete protease inhibitor cocktail (Roche, Sant Cugat del Valles, Spain). Quantification of protein concentration was determined using Lowry assay (DC protein assay, Bio-Rad). Thirty μg of protein were prepared in RIPA buffer 1× with loading buffer 1× and Sample reducing agent 1× and run in NuPAGE 4-12% Bis-Tris gels during 1 h at 150V at RT. Gels were transferred to iBlot Gel Transfer Stacks PVDF membranes (Life Technologies, Thermo Fisher Scientific) during 1:30 h at 110V at 4° C. Membranes were incubated with blocking solution (Tris-buffered saline with Tween-20 (TBS-T) with 5% bovine serum albumin) for 1 h at RT, and then incubated overnight at 4° C. with the indicated primary antibodies: anti-CCND1 (1:1000 Cell Signaling; ab134175), anti-CHAF1A (1:1000 Cell Signaling; #5480S), p 27 (1:1000 Cell Signaling; #3686) and phospho-Rb (pRB) (1:1000 Cell Signaling; #8516). Next, membranes were incubated with peroxidase-conjugated secondary antibodies for 1:30 h with anti-rabbit IgG-Peroxidase antibody produced in goat (1:10,000, Sigma-Aldrich; #A0545). Anti-actin HRP (1:40,000 Santa Cruz; sc-1616) were used as loading controls. Membranes were finally developed with EZ-ECL Chemiluminescence detection kit (Biological Industries, Kibbutz Beit-Haemek, Israel). Quantification of western blots were performed with ImageJ3. Each analysed protein band intensity was normalised to that of actin.

### Confocal Microscopy Imaging

SK-N-BE(2) cells were seeded in 8-wells Nunc Lab-Tek chamber slides 48 h before imaging (Thermofisher, USA). Cells were incubated with QS₄-Dil-miRNA (Cy5) complexes for 30 minutes and, then, the cell media was changed in order to remove the non-internalized complexes. The miRNA used was the same miRNA Control 1 used previously but functionalized; instead of with a Dy547, with Cy5 at the 5′end of the sense chain of the microRNA. The QS₄-Dil (^{Dil}QS₄) was formed as indicated previously in example 1. Confocal images were acquired using a LSM 800 microscope (Zeiss, Germany) after 2 minutes, 30 minutes or at indicated times for overnight incubation. Bright field images were obtained using a 488 nm laser. Dil and Cy5 fluorophores were excited using a 530 nm and 633 nm laser respectively and their signal collected from 550-620 nm and from 640-750 nm, respectively. Dil and Cy5 signals were collected in two different channels and processed in order to remove the cross talk between them. Images of complexes were processed to obtain the variation of the FRET ratio over time. For FRET ratio graph, each system was represented as the mean±SEM of technical triplicates in triplicate.

### Statistical Analysis

Unless otherwise stated, figures represent the average±SEM values of the mean of three independent experiments. Statistical significance was determined by unpaired two-tailed Student's t-test (GraphPad Prism Software, USA). * means p<0.05, means p<0.01 and *** means p<0.001.

### Results:

miRNA:

miRNA had to be transfected with QS systems to increase the miR-323a expression levels in SK-N-BE(2) cells, due to miRNA naked or miRNA complexed with MKC micelles could not increase the miRNA expression levels by qPCR (FIG. 10).

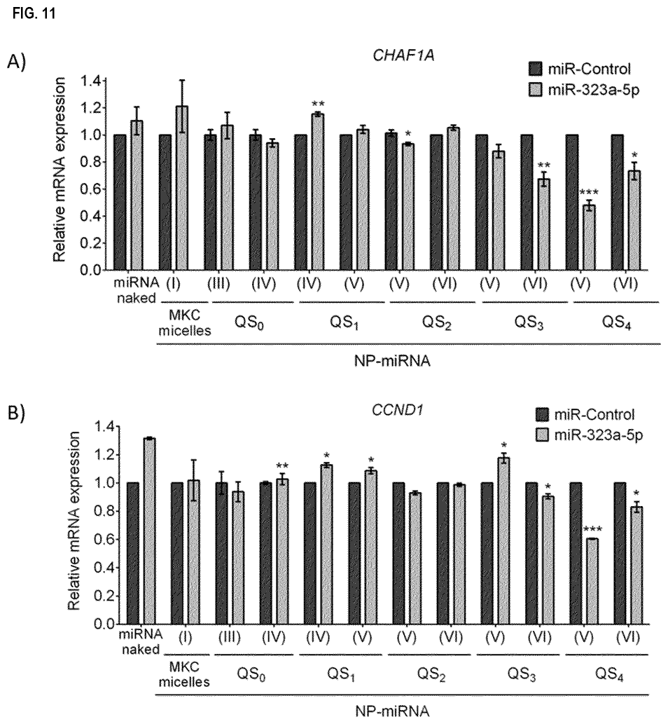

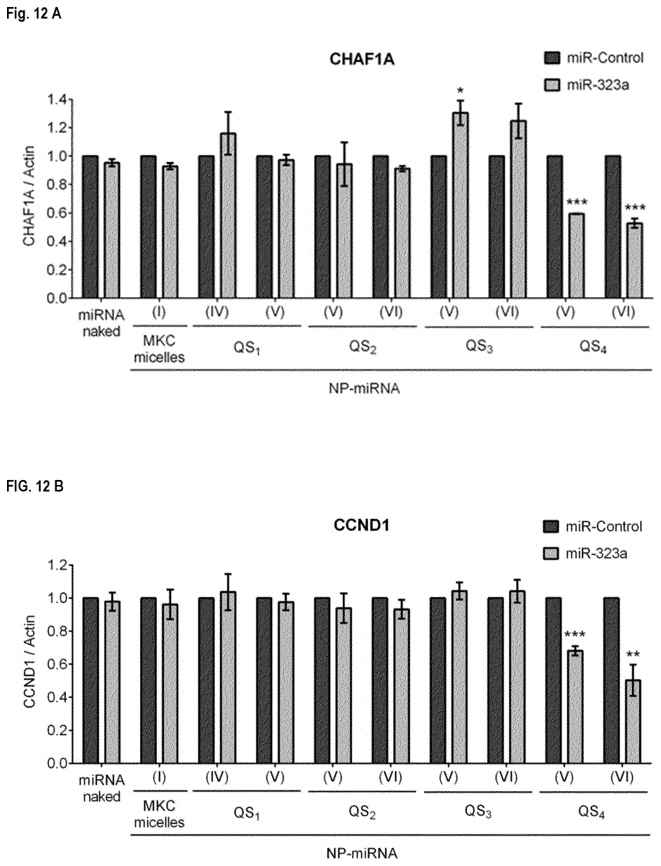

miR-323a targeted modification by qPCR and Western Blot (FIGS. 11-12): only when miR-323a-5p was transfected with QS₄ there were a reduction in miR-323a targets expression (CHAF1A and CCND1) at RNA (FIG. 11) and protein level (FIG. 12). QS₁₋₂ allowed the miRNA internalization but not the miRNA release.

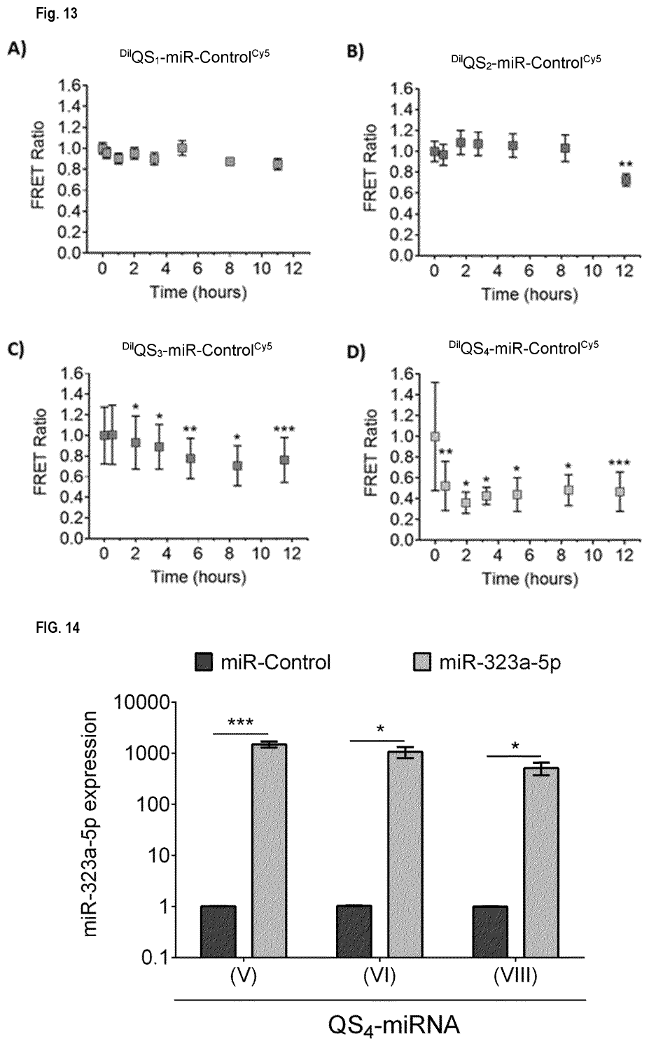

With QS₃ there were a reduction in miR-323a targets expression (CHAF1A and CCND1) at mRNA level. Whereas, QS₄ modified miR-323a-5p targets consistently at mRNA and protein level, QS₃ only reduced CHAF1A and CCND1 at mRNA with loading QS₃-miRNA (VI). These results were explained by the different amount of miRNA released and the required time for the miRNA to be released from QS₃ compared with QS₄. In QS₃-miRNA complexes, miRNA was released at slower pace than in QS₄-miRNA complexes (see FIG. 13).

QS-miR-323a complexes transfection allowed the increase of miR-323a-5p expression levels even in at high loadings of miRNA in QS₄ ((V to VIM)) (FIG. 14). miR-323a-5p transfected with QS₄ at miRNA-to-QS mass ratios (medium loadings), such as QS₄-miRNA (V) and (VI) modified direct miR-323a targets expression, such as CCND1 and CHAF1A, at mRNA and protein level. However, at miRNA-to-QS mass ratios (at high loadings) such as QS₄-miRNA (VIII) there were not miR-323a targets modification.

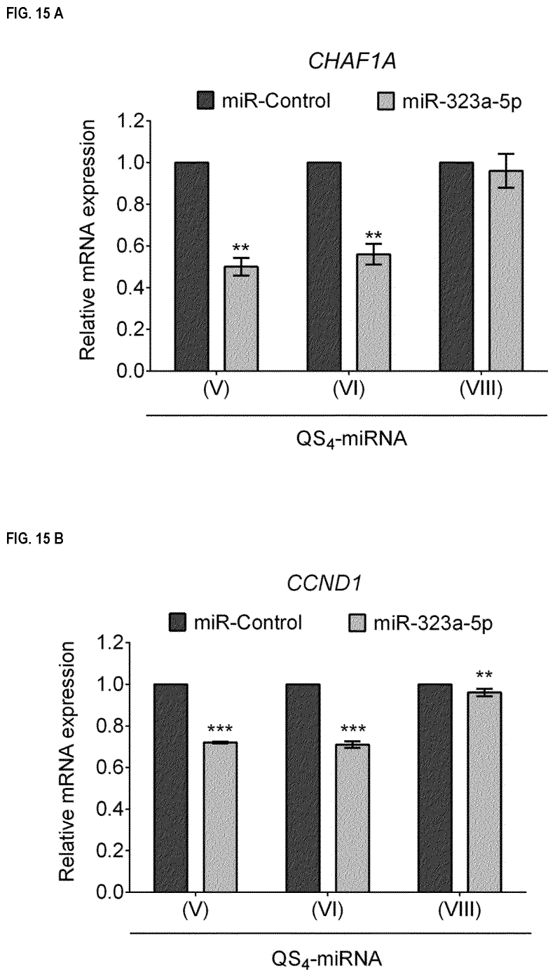

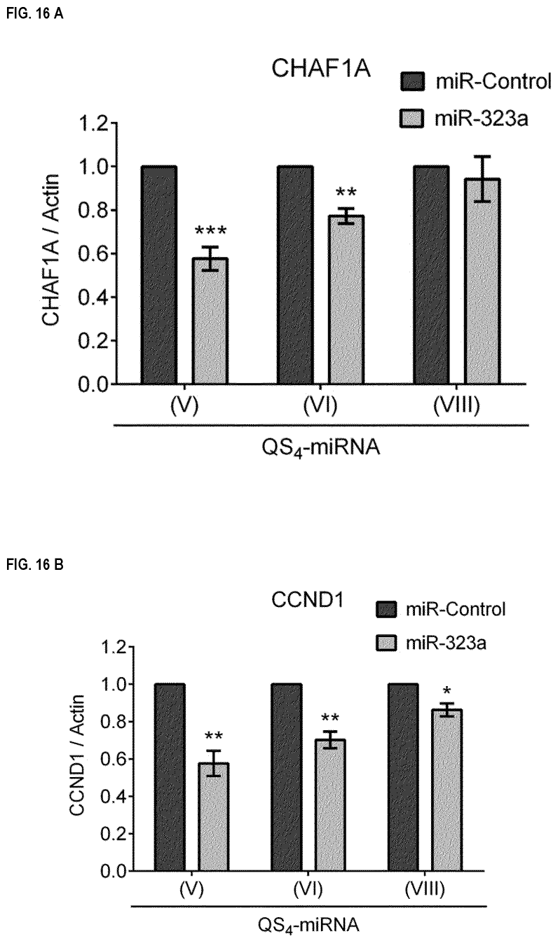

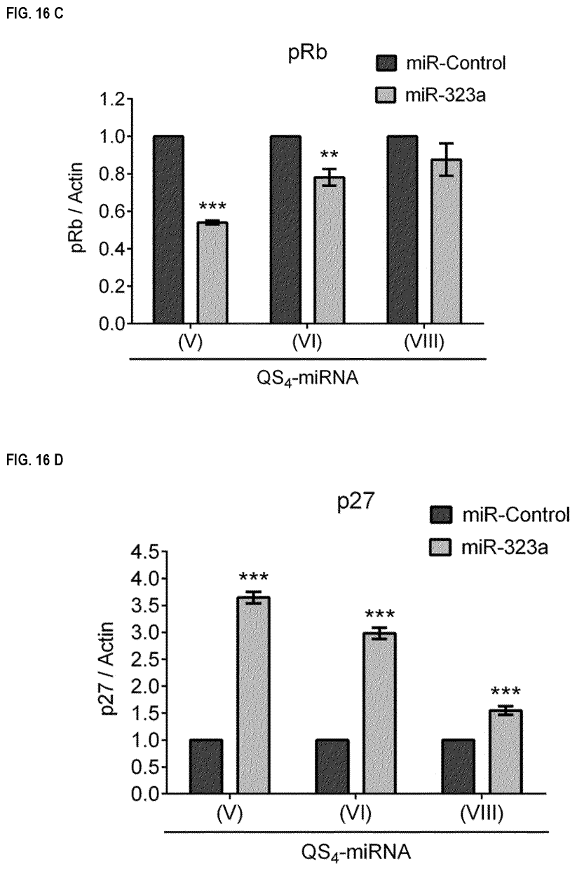

Moreover, miR-323a-5p transfected with QS₄ at different loadings (miRNA-to-QS mass ratios) such as QS₄-miRNA (V) and (VI) modified indirect targets of miR-323a, such as phospho-Rb (pRb) and p27, at mRNA (FIG. 15) and protein level (FIG. 16A, CHAF1A; FIG. 16B, CCND1; FIG. 16C, pRb; FIG. 16 D, p27).

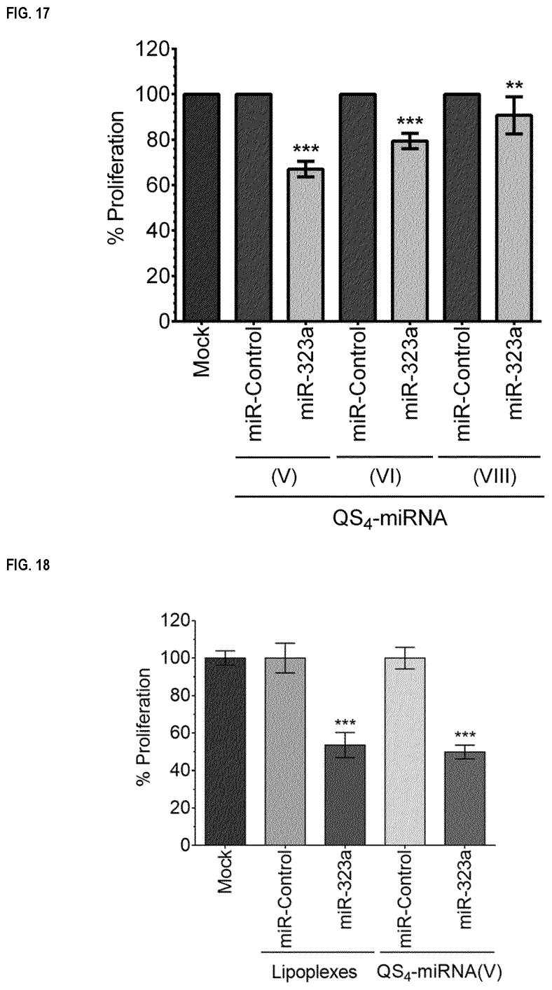

Overexpression of QS₄-miR-323a-5p complexes reduced SK-N-BE(2) cell proliferation after 96 hours, when miRNA was transfected with QS₄ at miRNA-to-QS mass ratios (loadings), such as (V) and (VI) (FIG. 17).

When miR-323a-5p was high, CCND1 expression was lost and pRB was not be phosphorylated, thus inhibiting cell cycle progression. Furthermore, p27 levels were thus increased and helped to inhibit the function of the CDK4/6 complex.

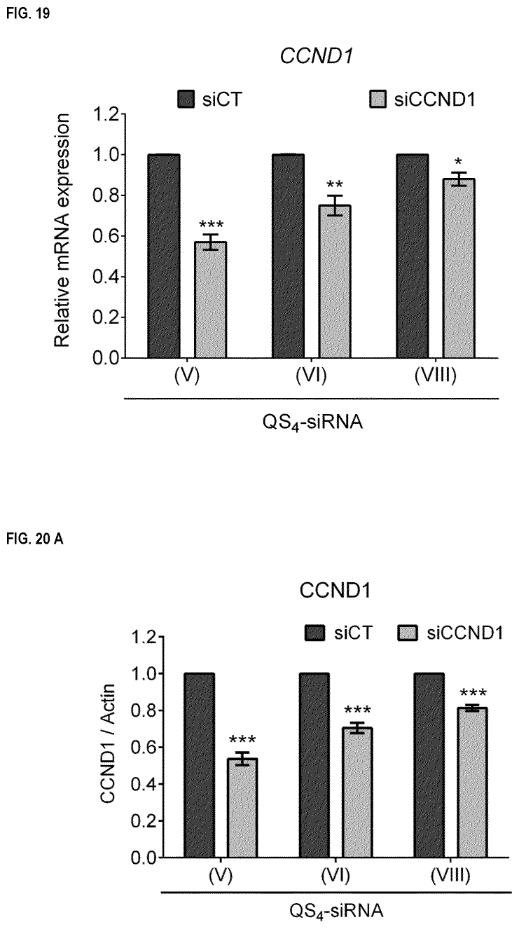

In addition, proliferation analysis in SK-N-BE(2) cells after transfection with QS₄-miR-323a-5p complexes (QS₄-miRNA (V)) compared to Lipofectamine2000® showed similar results (reduction p<0.001***, compared with control). Proliferation experiments were performed comparing miR-323a-5p versus miR-Control (50 nM) conjugated with QS₄ or liposomes (i.e. Lipofectamine 2000) in NB cells at 96 h post-transfection (FIG. 18).

siRNA:

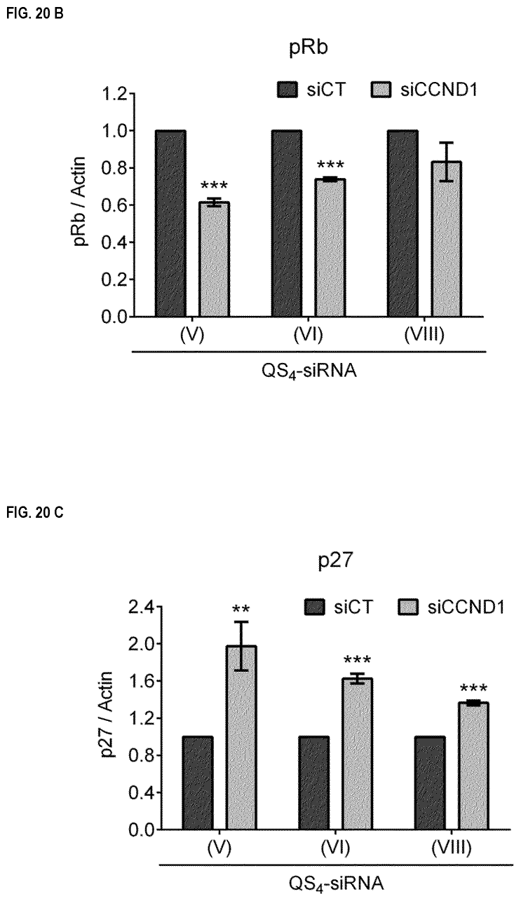

siCCND1 transfected with QS₄ at different loadings of QS₄-siRNA (siRNA-to-QS mass ratios), such as (V) and (VI) reduced CCND1 expression at mRNA (FIG. 19) and protein level (FIG. 20A). However, high loadings (siRNA-to-QS mass ratio) of QS₄-siRNA, such as loading (VIII) did not reduce CCND1 expression as well as the other loadings QS₄-siRNA (siRNA-to-QS mass ratios) (V) and (VI).

Moreover, siCCND1 transfected with QS₄ at loadings (siRNA-to-QS mass ratios) (V) and (VI) modified indirect targets of CCND1, such as pRb and p27, at mRNA or protein level (see FIG. 20B and FIG. 20C respectively). CCND1 depletion mirrored the best miR-323a-5p overexpression, not only the general effects on cell proliferation, but also on the reduction in phospho-Rb (pRb) levels and p27 accumulation.

Overexpression of QS₄-siCCND1 complexes reduced SK-N-BE(2) cell proliferation after 96 hours, when QS₄-siRNA was transfected at loadings (siRNA-to-QS mass ratios) (V) and (VI) (FIG. 21).

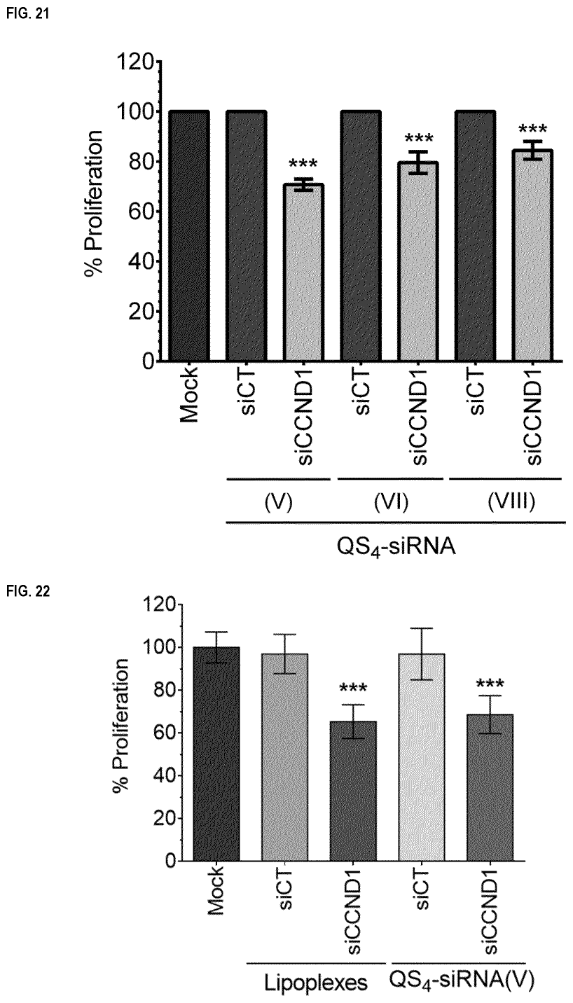

In addition, cell proliferation analysis in SK-N-BE(2) cells after transfection with QS₄-siCCND1 complexes (QS₄-siRNA (V)) compared with Lipofectamine2000® showed similar results (reduction of p<0.001***, compared with control). Proliferation experiments were performed comparing siCCND1 versus siControl (50 nM) complexed with QS₄ in NB cells at 96 h post-transfection (FIG. 22).

### Functionalization Studies:

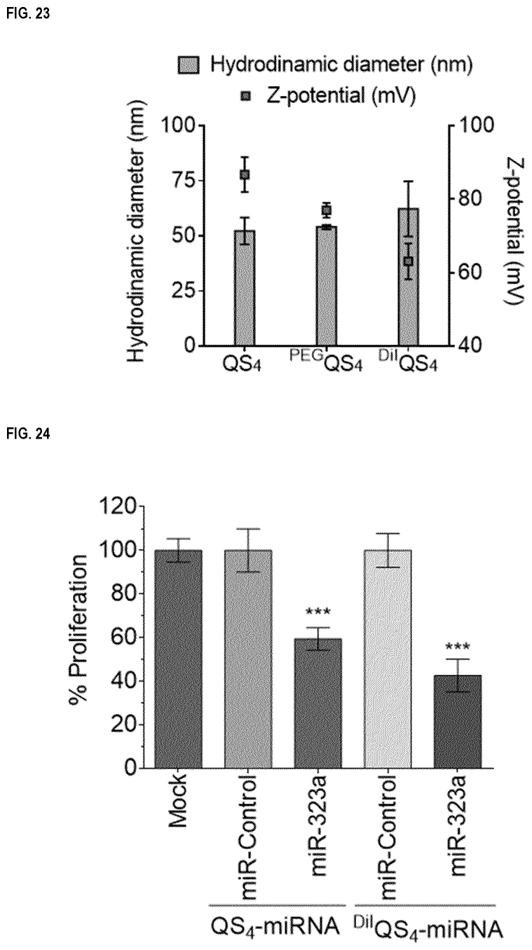

QS₄ was functionalized with Dil fluorophore or replacing 10% of DC-Chol sterol for Chol-PEG₁₀₀₀ polymer in the nanovesicles membrane as explained in example 1. QS₄-Dil (^{Dil}QS₄) and PEG-QS₄ functionalized QS presented similar size (30-70 nm), spherical shape, colloidal stability and surface positive charge like QS₄ (see FIG. 23). QS₄-miRNA and QS₄-(Dil)miRNA complexes presented similar morphology at the same loading of miRNA in QS.

QS₄ with or without Dil/PEG functionalization present a fully complexation efficiency of miRNA with a 100% of efficiency even at high miRNA-to-QS mass ratios (loadings QS-miRNA), such as (VIII) for ^{Dil}QS₄ and (VI) for PEG-QS₄. Moreover, decomplexation of QS-miRNA with SDS allowed almost 100% of miRNA release from QS.

By confocal imaging of live SK-N-BE(2) cells it was observed that QS₄-(Dil)miR-Control (miR-Control 1) complexes internalized in SK-N-BE(2) cells in a short time: after 30 minutes of QS₄-(Dil) miR-Control transfection.

FRET ratio experiments: QS₄-miRNA complexes remained stable for internalization time in cellular media. miRNA (Cy5) (miR-Control^{cy5}) and QS₄-Dil present a high FRET ratio efficiency owing the QS₄-Dil-miRNA (Cy5) attachment.

miRNA was released from ^{Dil}QS₃ after over-night incubation in cellular media at slow pace, while was released in less than 2 h from QS₄. miR-Control^{Cy5} and ^{Dil}QS₄ presented a low FRET ratio efficiency owing the miR-Control^{Cy5} and ^{Dil}QS₄ separation (see FIG. 13).

miRNA was released from QS₄-Dil after over-night incubation in cellular media. miRNA (Cy5) and QS₄-Dil presented a low FRET ratio efficiency owing the miRNA (Cy5) and QS₄-Dil separation.

Also, overexpression of ^{Dil}QS₄-miR-323a-5p at loading (VI) reduced SK-N-BE(2) cell proliferation after 96 hours, as much as or with higher effects than plain QS₄-miR-323a-5p.

See FIG. 24 for the cell proliferation analysis of SK-N-BE(2) cells transfected with ^{Dil}QS₄-miR-323a-5p complexes and plain QS₄-miR-323a-5p complexes.

QS₃-miRNA complexes and QS₄-miRNA complexes can be functional at loadings (miRNA-to-QS mass ratios) (IV) to (VII) depending on cell type and cell confluence. Moreover, QS₃-miRNA complexes required more than 48 h to induce miRNA targets modification at protein level, i.e. 72 h.

### Example 6: miRNA Protection from RNAse a Degradation after QS₄ Complexation

### Materials and Methods:

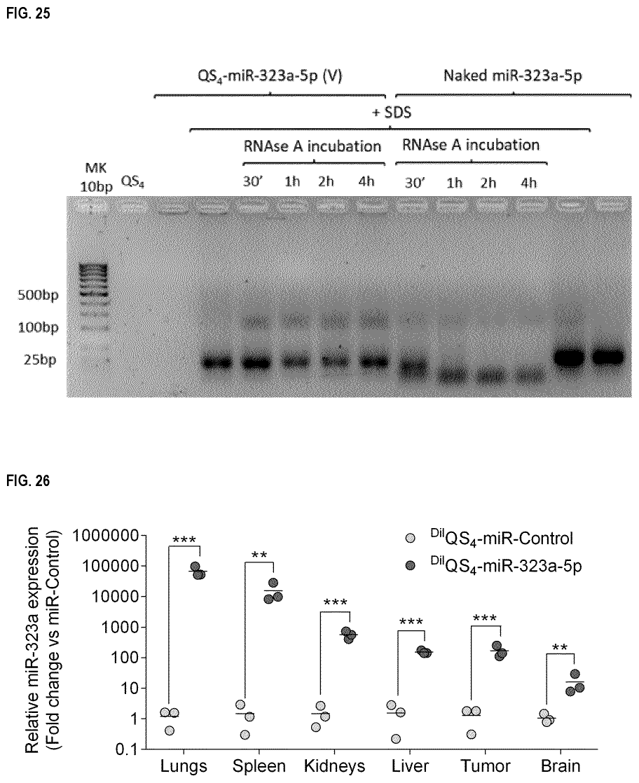

Agarose electrophoresis gels and QS₄-miRNA complexes were prepared as was explained before. To check QS capacity to protect miRNA from RNAse A degradation compared to naked miRNA, both complexes (FIG. 25; lane 5-8) and miRNA naked (FIG. 25; lane 9-12) were treated with 25 μg/mL of RNAse A for thirty minutes, one, two and four hours in a water bath at 310K. After that, the selected complexes (FIG. 25; lane 4-8) and miRNA naked (FIG. 25; lane 9-13) were treated with SDS 0.25% to ensure the release of the miRNA not degraded by the RNAse A. Then, all the preparations were loaded in the gel in PBS loading buffer (glycerol 0.008%). Finally, gels were run and images were taken. Agarose gel experiments were done in duplicate and a representative image is shown.

### Results:

QS₄ formulation showed the capacity to protect miRNA from ribonuclease-mediated degradation after four hours of RNAse A incubation in supraphysiological conditions (>1 μg/mL) (FIG. 25). After SDS addition the miRNA was not degraded by RNAse A, and could be released from QS₄ and detected in the agarose gel. So, QS₄ protected miR-323a-5p from degradation (lane 5-8) which may increase the miRNA half-life in in vivo circulation compared to naked miRNA which presented a short half-life in circulation (thirty minutes; lane 9-12).

### Example 7: In Vivo Experiments: Tissue Biodistribution of ^{Dil}QS₄:miRNA Complexes in Xenografts Mice Models

### Materials and Methods:

The QS were prepared as indicated in previous sections. For in vivo experiments, to form QS-sRNA complexes, the appropriate volume of QS₄ was added in a new Eppendorf to achieve a final concentration of 2.7 and 1.8 mg/mL of QS₄ per each injection of 200 μL for loading (V) and (VI) respectively. Then, 42.6 μL of sRNA were added over the appropriate volume (μL) of QS solution to obtain the desired sRNA-to-QS mass ratios (w/w), and maintaining a constant sRNA concentration (see Table 11). To achieve a constant final concentration of sRNA (i.e. 21.3 μM), QS-sRNA complexes were diluted with PBS 1× until reach the desired final volume (i.e. 200 μL), then mixed vigorously by vortexing and pipetting twice up-down (less than five minutes of incubation). The resulting QS-sRNA complexes were generated by ionic interactions between the positive charges on the surface of QS and the negative charges of sRNA. The different sRNA-to-QS mass ratios (w/w) were calculated between the QS mass and sRNA mass.

TABLE 11
Loadings of QS-sRNA complexes prepared at different sRNA-to-QS mass ratios to achieve a final
volume of i.e. 200 μL for in vivo experiments. sRNA used were miRNA or siRNA.
QS₄ refers to plain QS or functionalized with Dil.

| Mass ratio | | | | | | |
|---|---|---|---|---|---|---|
| sRNA/QS₄ | [QS₄ | | [sRNA | | Mass ratio | Mass ratio |
| loadings of | stock] | Volume of | stock] | Volume of | miRNA/QS₄ · | siRNA/QS₄ · |
| QS₄-sRNA) | (mg/mL) | QS (μL) | (μM) | sRNA (μL) | 10⁻² | 10⁻² |
| QS₄-sRNA (V) | 12.7 | 43.09 | 100 | 42.6 | 13.53 | 12.77 |
| QS₄-sRNA (VI) | | 28.72 | | | 20.31 | 19.15 |

SK-N-BE(2) cells (5×10⁶) were injected into the right flank of 6 to 8 week-old female athymic nude-Foxn1nu mice (n=3 mice/condition) in 300 μL of PBS:Matrigel (1:1). Tumor volume was measured every 2-3 days. Once tumors were ˜100-200 mm³, mice were randomized in two groups. Mice were injected with 2 mg/Kg of miR-Control (n=3) or miR-323a-5p conjugated with ^{Dil}QS₄ (n=3). After 24 hours, liver, lungs, brain, spleen, kidneys and tumor were removed and weighted. Tissues were homogenized with Bead-Ruptor 12 (Omni International; Georgia, USA) homogenizer (twenty seconds at speed 5 mA; two-three cycles until completely homogenisation) and the total RNA were extracted using the protocol explained before. Mature miRNA expression analysis was quantified by qPCR as was explained before. These results were plotted as the mean±SEM of three independent mice.

### Results:

^{Dil}QS₄ formulation showed the capacity to increase the miR-323a-5p expression in lungs, spleen, kidneys, liver and subcutaneous neuroblastoma tumors of mice with a higher increase of 150-, 66000-, 15000-, 570-, 150- and 125-fold change, respectively, compared with ^{Dil}QS₄-miR-Control (see FIG. 26).

No macroscopic signs of toxicity or adverse side effects were observed.

### CITATION LIST

### Patent Literature

WO2006079889
WO2017147407

### Non Patent Literature

Bumcrot D et al. Nat Chem Biol 2006, 2:711-719.
Grimaldi N. et al. Chem Soc Rev 2016, 45:6520-6545.
Cabrera I, et al. 2013 Nano Letters, 2013, 13(8), 3766-3774.
Ferrer-Tasies et al. Langmuir. 2013, 29(22):6519-28
Livak K J, Schmittgen T D. Methods 2001; 25: 402-408.
Cano-Sarabia M et al. Langmuir 2008, 24:2433-2437.
Elizondo E et al. Nanomed. 2012, 7:1391-1408.
Ardizzone et al, SMALL, 2018, 14 (16) DOI: 10.1002/smll.201703851.
Danaei, M.; et al. “Impact of Particle Size and Polydispersity Index on the Clinical Applications of Lipidic Nanocarrier Systems” Pharmaceutics 2018, 10, 57.
For reasons of completeness, various aspects of the invention are set out in the following numbered clauses:
   Clause 1. A nanovesicle comprising a sterol and a non-lipid cationic surfactant, wherein the sterol comprises DC-cholesterol (DC-Chol).
   Clause 2. The nanovesicle of clause 1, wherein the non-lipid cationic surfactant is of quaternary ammonium type.
   Clause 3. The nanovesicle according of clause 2, wherein the non-lipid cationic quaternary ammonium surfactants is selected from the list consisting of: myristalkonium chloride (MKC), cetyl trimethylammonium bromide (CTAB), cetylpyridinium chloride (CPC), benzalkonium chloride (BAC), cetyl trimethylammonium chloride (CTAC), menzethonium chloride (BZT), stearalkonium chloride, cetrimide, benzyldimethyldodecylammonium chloride, and any combinations thereof.
   Clause 4. The nanovesicle of clause 3, which it is a quatsome, wherein the non-lipid cationic quaternary ammonium surfactant is MKC and the sterol is 100% DC-Chol, preferably at a molar ratio 1:1.
   Clause 5. The nanovesicle of any one of clauses 1 to 4, which it is spherical, unilamellar, homogeneous in size and stable.
   Clause 6. The nanovesicle of any one of clauses 1 to 5 which comprises a nucleic acid, preferably a miRNA, siRNA and/or shRNA.
   Clause 7. The nanovesicle of clause 6 wherein the miRNA is selected from the list consisting of: hsa-miR-323a-5p, hsa-miR-497, has-miR-380-5p, hsa-miR-892b, hsa-miR-654-5p, hsa-miR-885-3p, hsa-miR-193a-3p, hsa-miR-661, hsa-miR-491-3p, hsa-miR-193b-5p, hsa-miR-3150a-3p, hsa-miR-744-5p, hsa-miR-326, hsa-miR-665, hsa-miR-185-3p, hsa-miR-34b-5p, hsa-miR-138-2-3p, hsa-miR-4440, hsa-miR-450b-3p, hsa-miR-1180, hsa-miR-3140-3p, hsa-miR-4291, hsa-miR-30b-3p, hsa-miR-541-3p, hsa-miR-483-5p, hsa-miR-4292, hsa-miR-124-3p, hsa-miR-1207-5p, hsa-miR-193b-3p, hsa-miR-221-5p, hsa-miR-3913-3p, hsa-miR-5095, hsa-miR-891b, hsa-miR-1275, hsa-miR-299-3p, hsa-miR-149-3p, hsa-miR-132-5p, hsa-miR-509-3-5p, hsa-miR-3677-3p, hsa-miR-876-3p, hsa-miR-940, hsa-miR-4655-5p, hsa-miR-555, hsa-miR-342-5p, hsa-miR-3181, hsa-miR-3154, hsa-miR-5585-3p, hsa-miR-708-5p, hsa-miR-3135a, hsa-miR-4664-3p, hsa-miR-4289, hsa-miR-135a-3p, hsa-miR-522-5p, and any combinations thereof; or, alternatively, the siRNA is selected from the list consisting of: siCCND1, siCHAF1A, siINCENP, siKIF11, siCDC25A, siFADD siBCL-XL, and any combinations thereof.
   Clause 8. The nanovesicle of any one of clauses 6 or 7 which is further bound to an element selected from the group consisting of: a fluorophore, a peptide, a polymer, an inorganic molecule, a lipid, a monosaccharide, an oligossacharide, an enzyme, an antibody or fragment of an antibody, an antigen, and any combination thereof.
   Clause 9. A pharmaceutical composition comprising a therapeutically effective amount of the nanovesicle of any one of clauses 1 to 8 and a pharmaceutically acceptable excipient or vehicle.
   Clause 10. The nanovesicle of any one of clauses 1 to 8 or the pharmaceutical composition of clause 9 as a delivery system.
   Clause 11. The nanovesicle of any one of clauses 1 to 8 or the pharmaceutical composition of clause 9 for use as a medicament.
   Clause 12. The nanovesicle of any one of clauses 1 to 8 or the pharmaceutical composition of clause 9 for use in the treatment of human disease, preferably in the treatment of cancer.
   Clause 13. The nanovesicle or the pharmaceutical composition for use of clause 12 wherein the cancer is neuroblastoma.
   Clause 14. The use of the nanovesicle of any one of clauses 1 to 8 as a bioimaging tool.
   Clause 15. A process for the production of a nanovesicle of any one of of clauses 1 to 8 using the DELOS-SUSP methodology.

1. A nanovesicle comprising a sterol and a non-lipid cationic surfactant, wherein the sterol comprises DC-cholesterol (DC-Chol).

2. The nanovesicle according to claim 1, wherein the nanovesicle is a non-liposomal nanovesicle.

3. The nanovesicle according to claim 1, wherein the percentage of DC-Chol in respect to the sterol is at least 20%.

4. The nanovesicle according to claim 1, wherein the percentage of DC-Chol in respect to the sterol is at least 47%.

5. The nanovesicle according to claim 1, wherein the sterol is a mixture of DC-Chol and cholesterol, or, alternatively, a mixture of DC-chol and a cholesterol derivative.

6. The nanovesicle according to claim 5, wherein the cholesterol derivative comprises polyethylene glycol (PEG).

7. The nanovesicle according to claim 6, wherein the cholesterol derivative is Chol-PEGn-X, wherein “n” is the length of the PEG chain; and wherein “X” is —SH, —OH, —CHO, —OCH₃, —NH₂, —NH, —CH₃, —N₃, —COOH, -maleimide, a peptide, and antibody or a sugar.

8. The nanovesicle according to claim 7, wherein the peptide is selected from the list consisting of: a GD2 mimic binding peptide, a neuropeptide Y; a peptide comprising the sequence SEQ ID NO: 22, a P75 neurotrophin receptor, a Rabies virus glycoprotein (RVG) peptide, a dopaminergic peptide, a RGD-peptide, and a GD2 antibody.

9. The nanovesicle according to claim 8, wherein the peptide is selected from the list consisting of: SEQ ID NO: 22, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, and a RGD-peptide.

10. The nanovesicle according to claim 7, wherein the sugar is D-glucose or a glucosamine derivative.

11. The nanovesicle according to claim 1, wherein the non-lipid cationic surfactant is of quaternary ammonium type.

12. The nanovesicle according to claim 1, wherein the non-lipid cationic quaternary ammonium surfactants is selected from the list consisting of: myristalkonium chloride (MKC), cetyl trimethylammonium bromide (CTAB), cetylpyridinium chloride (CPC), benzalkonium chloride (BAC), cetyl trimethylammonium chloride (CTAC), menzethonium chloride (BZT), stearalkonium chloride, cetrimide, benzyldimethyldodecylammonium chloride, and any combinations thereof.

13-14. (canceled)

15. The nanovesicle according to claim 12, which it is a quatsome, wherein the non-lipid cationic quaternary ammonium surfactant is MKC and the sterol is 100% DC-Chol, preferably at a molar ratio 1:1.

16. The nanovesicle according to claim 1, which it is spherical, unilamellar, homogeneous in size and stable.

17. The nanovesicle according to claim 16, wherein the nanovesicle has a mean diameter smaller than 300 nm, a polydispersity index (PDI) of 0.1-0.3, and is stable at least up to 2 months.

18. The nanovesicle according to claim 1 which comprises a nucleic acid, preferably a miRNA, siRNA and/or shRNA.

19. The nanovesicle according to claim 18 wherein the miRNA is selected from the list consisting of: hsa-miR-323a-5p, hsa-miR-497, has-miR-380-5p, hsa-miR-892b, hsa-miR-654-5p, hsa-miR-885-3p, hsa-miR-193a-3p, hsa-miR-661, hsa-miR-491-3p, hsa-miR-193b-5p, hsa-miR-3150a-3p, hsa-miR-744-5p, hsa-miR-326, hsa-miR-665, hsa-miR-185-3p, hsa-miR-34b-5p, hsa-miR-138-2-3p, hsa-miR-4440, hsa-miR-450b-3p, hsa-miR-1180, hsa-miR-3140-3p, hsa-miR-4291, hsa-miR-30b-3p, hsa-miR-541-3p, hsa-miR-483-5p, hsa-miR-4292, hsa-miR-124-3p, hsa-miR-1207-5p, hsa-miR-193b-3p, hsa-miR-221-5p, hsa-miR-3913-3p, hsa-miR-5095, hsa-miR-891b, hsa-miR-1275, hsa-miR-299-3p, hsa-miR-149-3p, hsa-miR-132-5p, hsa-miR-509-3-5p, hsa-miR-3677-3p, hsa-miR-876-3p, hsa-miR-940, hsa-miR-4655-5p, hsa-miR-555, hsa-miR-342-5p, hsa-miR-3181, hsa-miR-3154, hsa-miR-5585-3p, hsa-miR-708-5p, hsa-miR-3135a, hsa-miR-4664-3p, hsa-miR-4289, hsa-miR-135a-3p, hsa-miR-522-5p, and any combinations thereof; or, alternatively, the siRNA is selected from the list consisting of: siCCND1, siCHAF1A, siINCENP, siKIF11, siCDC25A, siFADD siBCL-XL, and any combinations thereof.

20. (canceled)

21. A pharmaceutical composition comprising a therapeutically effective amount of the nanovesicle as defined in claim 1 and a pharmaceutically acceptable excipient or vehicle.

22-23. (canceled)

24. A method for the treatment or prevention of cancer, that comprises administering a therapeutically effective amount of the nanovesicle as defined in claim 1 together with pharmaceutically acceptable carriers or excipients to a subject in need of it.

25. The method according to claim 24 wherein the cancer is neuroblastoma.

26-27. (canceled)
